(19) United States
(12) Patent Application Publication
(10) Pub. No.: US 2009/0220444 A1
(43) Pub. Date: Sep. 3, 2009

(54) Antiperspirant and Deodorant Compositions with Improved Protective Action

(75) Inventors: Gertraud Teckenbrock, Sprockhoevel (DE); Barbara Heide, Krefeld (DE)

Correspondence Address: Ratner Prestia, P.O. Box 980, Valley Forge, PA, 19482, US

(73) Assignee: Henkel AG & Co. KGaA, Duesseldorf (DE)

(21) Appl. No.: 12/394,558

(22) Filed: Feb. 27, 2009

Related U.S. Application Data
(63) Continuation of application No. PCT/EP2007/058644, filed on Aug. 21, 2007.

(30) Foreign Application Priority Data
Aug. 29, 2006 (DE) 10 2006 040 302.9

(51) Int. Cl. A61K 8/28; A61K 8/26; A61P 17/00

(52) U.S. Cl. 424/66; 424/68

(57) ABSTRACT

Cosmetic and dermatological antiperspirant and deodorant compositions which have an improved care effect and comprise at least one yoghurt protein and/or at least one hydrolysate of a yoghurt protein and/or at least one N—C₂-C₂₄-acyl derivative and/or at least one C₂-C₂₄ ester and/or at least one physiologically tolerated salt of these substances.

### CROSS-REFERENCE TO RELATED APPLICATIONS

This application is a continuation under 35 U.S.C. §§ 120 and 365(c) of International Application PCT/EP2007/058644, filed on Aug. 21, 2007. This application also claims priority under 35 U.S.C. § 119 of DE 10 2006 040 302.9, filed on Aug. 29, 2006. The disclosures of PCT/EP2007/058644 and DE 10 2006 040 302.9 are incorporated herein by reference in their entirety.

### BACKGROUND OF THE INVENTION

The present application relates to cosmetic and dermatological antiperspirant and deodorant compositions which exhibit an improved protective action and/or a reduced irritation potential.

Conventional commercial antiperspirant compositions, hereinafter also denoted antiperspirants, contain at least one water-soluble astringent inorganic and organic salt of aluminum, zinc or selected aluminum-zirconium mixed salts as the antiperspirant active ingredient. Conventional commercial deodorant compositions, hereinafter also denoted deodorants, frequently contain at least one antibacterial active ingredient as the deodorant active ingredient.

The antiperspirant active ingredients have no direct influence on the activity of the sweat glands, but instead minimize secretion of sweat by constricting the outflow ducts. On treated areas of skin, the Al salts bring about an antiperspirant action by superficial blocking of the sweat gland ducts as a result of Al-mucopolysaccharide precipitates.

Antiperspirant and deodorant compositions are conventionally applied and used in the armpit area.

Due to its position and function, the skin of the armpit has a weak barrier action. The skin in the armpit area is thus more sensitive than the rest of the skin on the body, frequently at least just as sensitive as the skin of the face. Many consumers consider hairless armpit skin to be more attractive and more conducive to hygiene, since hair growth enlarges the sweat-bearing area in the armpit area and so contributes to increased development of body odor. As a consequence, regular shaving of the armpit hair is part of their personal hygiene routine. Shaving the armpits irritates the skin mechanically and additionally weakens its barrier action. After shaving, an antiperspirant and/or deodorizing cosmetic preparation, for example a deodorant spray or stick, is conventionally applied onto the armpit skin. The antiperspirant and/or deodorizing active ingredients usually exhibit, for example due to their acidic pH value, a considerable irritation potential which, on the mechanically irritated armpit skin can cause erythema, an unpleasant stinging, feeling of tightness and/or itching.

### DESCRIPTION OF THE INVENTION

There is accordingly a constant need for particularly skin compatible formulations which are suitable as carriers for cosmetic and dermatological antiperspirant and deodorant compositions. A need furthermore remains for particularly skin compatible formulations which, as carriers for cosmetic and dermatological antiperspirant and deodorant compositions, may be capable of making the antiperspirant and deodorant active ingredients, which occasionally have an irritant action on the skin when used regularly and/or on mechanically or chemically irritated skin, more skin compatible or of moderating their irritant action.

The problem addressed by the present invention was to identify active ingredients and/or carriers for cosmetic and dermatological antiperspirant and deodorant compositions with particularly elevated skin compatibility. A further problem addressed by the present invention was to identify active ingredients and/or carriers for cosmetic and dermatological antiperspirant and deodorant compositions with particularly elevated skin compatibility in particular for mechanically irritated skin. A further problem addressed by the present invention was to identify active ingredients and/or carriers for cosmetic and dermatological antiperspirant and deodorant compositions which are capable of making antiperspirant and deodorant active ingredients which have an irritant action on the skin more skin compatible or of moderating their irritant action. A further problem addressed by the present invention was to identify active ingredients and/or carriers for cosmetic and dermatological antiperspirant and deodorant compositions which reduce stinging and/or itching of the skin on application.

It has surprisingly been found that active ingredients selected from yoghurt proteins or yoghurt protein derivatives, including yoghurt protein hydrolysates, N—C₂-C₂₄ acyl derivatives and C₂-C₂₄ esters of yoghurt proteins, and the physiologically acceptable salts of these protein active ingredients solve the stated problem.

The present application accordingly provides a cosmetic or dermatological deodorant or antiperspirant composition containing in a cosmetically or dermatologically acceptable carrier at least one deodorant or antiperspirant active ingredient, at least one yoghurt protein and/or at least one yoghurt protein derivative, such as at least one hydrolysate of a yoghurt protein and/or at least one N—C₂-C₂₄ acyl derivative and/or at least one C₂-C₂₄ ester, and/or at least one physiologically acceptable salt of these protein active ingredients, and mixtures of these protein active ingredients.

The present application also provides the use of at least one yoghurt protein and/or at least one yoghurt protein derivative, such as at least one hydrolysate of a yoghurt protein and/or at least one N—C₂-C₂₄ acyl derivative and/or at least one C₂-C₂₄ ester, and/or at least one physiologically acceptable salt of these substances, and mixtures of these protein active ingredients, in a cosmetically or dermatologically acceptable carrier, containing at least one deodorant or antiperspirant active ingredient, for improving the skin compatibility and/or the care effect of the composition and/or for reducing or avoiding stinging of the skin, a feeling of tightness, itching and/or visible irritation, such as erythema, which is/are caused by contact of the skin with the composition.

The present application also provides the use of at least one yoghurt protein and/or at least one yoghurt protein derivative, such as at least one hydrolysate of a yoghurt protein and/or at least one N—C₂-C₂₄ acyl derivative and/or at least one C₂-C₂₄ ester, and/or at least one physiologically acceptable salt of these substances, and mixtures of these protein active ingredients in combination with at least one further active ingredient selected from monosaccharides having 5 or 6 carbon atoms and disaccharides, in a cosmetically or dermatologically acceptable carrier, containing at least one deodorant or antiperspirant active ingredient, for improving the skin compatibility and/or the protective action of the composition and/or for reducing or avoiding stinging of the skin, a feeling of tightness, itching and/or visible irritation, such as erythema, which is/are caused by contact of the skin with the composition.

A preferred embodiment of the invention is characterized in that the at least one yoghurt protein, at least one hydrolysate of a yoghurt protein and/or the at least one N—C₂-C₂₄ acyl derivative and/or the at least one C₂-C₂₄ ester and/or the at least one physiologically acceptable salt of these substances is contained in the form of yoghurt.

A further preferred embodiment of the invention is characterized in that the at least one yoghurt protein, at least one hydrolysate of a yoghurt protein and/or the at least one N—C₂-C₂₄ acyl derivative and/or the at least one C₂-C₂₄ ester and/or the at least one physiologically acceptable salt of these substances is contained in the form of dried yoghurt, in particular in the form of spray-dried yoghurt, freeze-dried yoghurt or lyophilized yoghurt. It is likewise preferred according to the invention for the yoghurt contained therein to be dried in a manner other than that listed above.

A hydrolysate of a yoghurt protein is taken according to the invention to mean an oligopeptide of at least three amino acids, preferably of at least 5 peptides, particularly preferably of at least 8 peptides, which is obtainable by hydrolysis from native yoghurt protein.

A further preferred embodiment of the invention is characterized in that an N—C₂-C₂₄ acyl derivative of a yoghurt protein or of a yoghurt protein hydrolysate is contained therein. The C₂-C₂₄ acyl residue, with which the at least one yoghurt protein or at least one yoghurt protein hydrolysate is derivatized on at least one amino group, is preferably selected from an acetyl, propanoyl, butanoyl, pentanoyl, hexanoyl, heptanoyl, octanoyl, nonanoyl, decanoyl, undecanoyl, lauroyl, tridecanoyl, myristoyl, pentadecanoyl, cetoyl, palmitoyl, stearoyl, elaidoyl, arachidoyl or behenoyl residue. Mixtures of C₈-C₁₈ acyl residues are also denoted “cocoyl residue” and are likewise preferred substituents. It is particularly preferred for a terminal amino group to be substituted with a C₂-C₂₄ acyl residue.

A further preferred embodiment of the invention is characterized in that, on those amino acids which bear a hydroxyl group, the at least one yoghurt protein or at least one yoghurt protein hydrolysate is linked with the above-stated C₂-C₂₄ acyl residues on at least one of these hydroxyl groups via an ester bond.

A further preferred embodiment of the invention is characterized in that the at least one yoghurt protein or at least one yoghurt protein hydrolysate, the N—C₂-C₂₄ acyl derivative thereof or the C₂-C₂₄ ester thereof is present in the form of at least one physiologically acceptable salt. Physiologically acceptable protein active ingredient salts which are preferred according to the invention are selected from ammonium, alkali metal, magnesium, calcium, aluminum, zinc and manganese salts. Sodium, potassium, magnesium, aluminum, zinc and manganese salts are particularly preferred according to the invention.

It has surprisingly been found that the yoghurt protein or yoghurt protein hydrolysate substituted with the above-stated C₂-C₂₄ acyl residues may exhibit a further improvement of antiirritant action relative to the unsubstituted yoghurt protein or yoghurt protein hydrolysate.

A further preferred embodiment of the invention is characterized in that the yoghurt protein, the yoghurt protein hydrolysate, the N—C₂-C₂₄ acyl derivative, the C₂-C₂₄ ester derivative and/or the physiologically acceptable salt thereof is contained in a total quantity of 0.001 to 0.1 wt. %, preferably of 0.005-0.07 wt. %, particularly preferably of 0.01-0.05 wt. % and extremely preferably of 0.02-0.04 wt. %, in each case relative to the total composition. Total quantities of yoghurt protein active ingredients of more than 0.1 to 1 wt. % may likewise be preferred according to the invention. Total quantities of yoghurt protein active ingredients of more than 0.1 to 1 wt. % may in particular be preferred according to the invention when the composition does not contain an aluminum salt.

A yoghurt protein raw material which is particularly preferred according to the invention is obtainable from Cosmetochem under the tradename “Yoghurt Protein GBU”.

Antiperspirant active ingredients which are preferred according to the invention are selected from among aluminum chlorohydrates, in particular the aluminum chlorohydrate of the general formula [Al₂(OH)₅Cl.2-3H₂O]ₙ, which may be present in unactivated or activated (depolymerized) form, furthermore aluminum sesquichlorohydrate, aluminum chlorohydrex propylene glycol (PG) or polyethylene glycol (PEG), aluminum sesquichlorohydrex PG or PEG, aluminum PG dichlorohydrex or aluminum PEG dichlorohydrex, aluminum hydroxide, furthermore selected from aluminum-zirconium chlorohydrates, such as aluminum-zirconium trichlorohydrate, aluminum-zirconium tetrachlorohydrate, aluminum-zirconium pentachlorohydrate, aluminum-zirconium octachloorhydrate, aluminum-zirconium chlorohydrate glycine complexes, such as aluminum-zirconium trichlorohydrex glycine, aluminum-zirconium tetrachlorohydrex glycine, aluminum-zirconium pentachlorohydrex glycine, aluminum-zirconium octachlorohydrex glycine, potassium aluminum sulfate (KAI(SO₄)₂.12H₂O, alum), aluminum undecylenoyl collagen amino acid, sodium-aluminum lactate+aluminum sulfate, sodium-aluminum chlorohydroxylactate, aluminum bromohydrate, aluminum chloride, the complexes of zinc and sodium salts, the complexes of lanthanum and cerium, the aluminum salts of lipoamino acids, aluminum sulfate, aluminum lactate, aluminum chlorohydroxyallantoinate, sodium-aluminum chlorohydroxylactate, zinc chloride, zinc sulfocarbolate, zinc sulfate and zirconium chlorohydrate. According to the invention, water solubility is taken to mean solubility of at least 5 wt. % at 20° C., i.e. that quantities of at least 5 g of the antiperspirant active ingredient are soluble in 95 g of water at 20° C. The antiperspirant active ingredients may be used as aqueous solutions.

Particularly preferred deodorant or antiperspirant compositions according to the invention are characterized in that the at least one antiperspirant active ingredient is contained in a total quantity of 3-25 wt. %, preferably of 5-22 wt. % and in particular of 10-20 wt. %, relative to the total weight of the active substance in the total composition.

The quantity of antiperspirant salt(s) stated in wt. % in the present patent application should be calculated using the US Pharmacopoeia (USP) method, according to which bound water of crystallization and other ligands, for example glycine, are excluded.

In one particularly preferred embodiment, the composition contains an astringent aluminum salt, in particular aluminum chlorohydrate, which is distributed, for example, in pulverulent form as Micro Dry® Ultrafine or Micro Dry® SUF by Reheis, in the form of an aqueous solution as Locron® L by Clariant, as Chlorhydrol® and in activated form as Reach® 103 or Reach® 501 by Reheis. Reheis offers an aluminum sesquichlorohydrate for sale under the name Reach® 301, which is likewise particularly preferred. It may be particularly preferred according to the invention to use aluminum-zirconium tetrachlorohydrex glycine complex, which is commercially available for example from Reheis under the name Rezal® 36G or Reach® 908. In a further particularly preferred embodiment, the compositions according to the invention may contain both at least one deodorant active ingredient and at least one antiperspirant active ingredient.

Deodorant active ingredients which are preferred according to the invention are odor absorbers, ion exchangers with a deodorizing action, bacteriostatic agents, prebiotically active components and enzyme inhibitors or, particularly preferably, combinations of the stated active ingredients.

Silicates serve as odor absorbers which also simultaneously advantageously assist the Theological properties of the composition according to the invention. Silicates which are particularly preferred according to the invention are above all phyllosilicates and among these in particular montmorillonite, kaolinite, ilite, beidellite, nontronite, saponite, hectorite, bentonite, smectite and talcum. Further preferred odor absorbers are for example zeolites, zinc ricinoleate, cyclodextrins, certain metal oxides, such as for example aluminum oxide, and chlorophyll. They are preferably used in a quantity of 0.1-10 wt. %, particularly preferably of 0.5-7 wt. % and extremely preferably of 1-5 wt. %, in each case relative to the total composition.

According to the invention, bacteriostatic or antimicrobial active ingredients are taken to mean those active ingredients which reduce the number of skin bacteria involved in odor formation or inhibit the growth thereof. These microbes inter alia include various species from the group of staphylococci, the group of corynebacteria, anaerococci and micrococci.

Bacteriostatic or antimicrobial active ingredients which are preferred according to the invention are in particular organohalogen compounds and organohalides, quaternary ammonium compounds, a range of plant extracts and zinc compounds. These include inter alia triclosan, chlorhexidine and chlorhexidine gluconate, 3,4,4′-trichlorocarbanilide, bromochlorophene, dichlorophene, chlorothymol, chloroxylenol, hexachlorophene, dichloro-m-xylenol, dequalinium chloride, domiphene bromide, ammonium phenolsulfonate, benzalkonium halides, benzalkonium cetyl phosphate, benzalkonium saccharinates, benzethonium chloride, cetylpyridinium chloride, laurylpyridinium chloride, laurylisoquinolinium bromide, methylbenzethonium chloride. Phenol, phenoxyethanol, disodium dihydroxyethyl sulfosuccinylundecylenate, sodium bicarbonate, zinc lactate, sodium phenolsulfonate and zinc phenolsulfonate, ketoglutaric acid, terpene alcohols such as for example farnesol, chlorophyllin copper complexes, α-monoalkyl glycerol ethers with a branched or linear saturated or unsaturated, optionally hydroxylated C₆-C₂₂ alkyl residue, particularly preferably α-(2-ethylhexyl) glycerol ether, commercially available as Sensiva® SC 50 (from Schülke & Mayr), carboxylic acid esters of mono-, di- and triglycerol (for example glycerol monolaurate, diglycerol monocaprate), lantibiotics and plant extracts (for example green tea and constituents of lime blossom oil) may also be used.

Further preferred deodorant active ingredients are selected from “prebiotically active” components, which according to the invention should be taken to mean those components which inhibit only or at least predominantly the odor-forming microorganisms of the skin microflora, but not the desired microbes, i.e. the non-odor-forming microbes which are part of the healthy skin microflora. This explicitly includes those active ingredients which are disclosed as prebiotically active in published patent applications DE 10333245 and DE 10 2004 011 968; these include conifer extracts, in particular from the Pinaceae group, and plant extracts from the group of Sapindaceae, Araliaceae, Lamiaceae and Saxifragaceae, in particular extracts from Picea spp., Paullinia sp., Panax sp., Lamium album or Ribes nigrum and mixtures of these substances. Further preferred deodorant active ingredients are selected from perfume oils with a bacteriostatic action and Deosafe® perfume oils which are obtainable from Symrise, formerly Haarmann and Reimer.

Enzyme inhibitors include substances which inhibit the enzymes responsible for breaking down sweat, in particular aryl sulfatase, β-glucuronidase, aminoacylase, esterases, lipases and/or lipoxygenase, for example trialkyl citric acid esters, in particular triethyl citrate, or zinc glycinate. Preferred deodorant or antiperspirant compositions according to the invention are characterized in that the at least one deodorant active ingredient is selected from aryl sulfatase inhibitors, β-glucuronidase inhibitors, aminoacylase inhibitors, esterase inhibitors, lipase inhibitors and lipoxygenase inhibitors, α-monoalkyl glycerol ethers with a branched or linear saturated or unsaturated, optionally hydroxylated C₆-C₂₂ alkyl residue, in particular α-(2-ethylhexyl) glycerol ether, phenoxyethanol, perfume oils with a bacteriostatic action, Deosafe® perfume oils (Deosafe® is a registered trademark of Symrise, formerly Haarmann & Reimer), prebiotically active components, trialkyl citric acid esters, in particular triethyl citrate, active ingredients which reduce the number or inhibit the growth of skin microbes involved in odor formation from the group of staphylococci, corynebacteria, anaerococci and micrococci, zinc compounds, in particular zinc phenolsulfonate and zinc ricinoleate, organohalogen compounds, in particular triclosan, chlorhexidine, chlorhexidine gluconate and benzalkonium halides, quaternary ammonium compounds, in particular cetylpyridinium chloride, odor absorbers, in particular silicates and zeolites, sodium bicarbonate, lantibiotics, and mixtures of the above-stated substances.

Further preferred deodorant or antiperspirant compositions according to the invention are characterized in that the at least one deodorant active ingredient is contained in a total quantity of 0.1-10 wt. %, preferably of 0.2-7 wt. %, particularly preferably of 0.3-5 wt. % and extremely preferably of 0.4-1.0 wt. %, in each case relative to the total weight of the active substance of the deodorant active ingredient or the deodorant active ingredients in the total composition.

In a further particularly preferred embodiment, the compositions according to the invention may contain both at least one deodorant active ingredient and at least one antiperspirant active ingredient.

A further preferred embodiment of the invention is characterized in that the composition according to the invention or composition used according to the invention contains at least one monosaccharide with 5 or 6 carbon atoms and/or at least one disaccharide.

It has surprisingly been found that the combination of yoghurt protein and/or at least one hydrolysate of a yoghurt protein and/or at least one N—C₂-C₂₄ acyl derivative and/or at least one C₂-C₂₄ ester and/or at least one physiologically acceptable salt of these substances, and mixtures of these protein active ingredients, with at least one monosaccharide with 5 or 6 carbon atoms and/or at least one disaccharide may exhibit a further improvement of the antiirritant action relative to the unsubstituted yoghurt protein or yoghurt protein hydrolysate.

A particularly preferred embodiment of the invention is characterized in that the monosaccharide with 5 or 6 carbon atoms is selected from glucose, fructose, galactose, arabinose, ribose, xylose, lyxose, allose, altrose, mannose, gulose, idose, talose, fucose and rhamnose. Of these, glucose, galactose, fructose, fucose and rhamnose, in particular glucose and galactose, are extremely preferred.

A further particularly preferred embodiment of the invention is characterized in that the disaccharide with 5 or 6 carbon atoms is selected from sucrose, lactose and trehalose. Of these, sucrose and lactose, in particular lactose, are extremely preferred.

A further particularly preferred embodiment of the invention is characterized in that at least one monosaccharide with 5 or 6 carbon atoms and/or at least one disaccharide is contained in a total quantity of 0.001 to 2.0 wt. %, preferably of 0.005-1.0 wt. %, particularly preferably of 0.01-0.5 wt. % and extremely preferably of 0.03-0.1 wt. %, in each case relative to the total composition.

A further particularly preferred embodiment of the invention is characterized by an active ingredient combination of yoghurt protein and lactose.

A further particularly preferred embodiment of the invention is characterized by an active ingredient combination of yoghurt protein hydrolysate and lactose.

A further particularly preferred embodiment of the invention is characterized by an active ingredient combination of yoghurt protein and galactose.

A further particularly preferred embodiment of the invention is characterized by an active ingredient combination of yoghurt protein hydrolysate and galactose.

A further particularly preferred embodiment of the invention is characterized by an active ingredient combination of yoghurt protein and glucose.

A further particularly preferred embodiment of the invention is characterized by an active ingredient combination of yoghurt protein hydrolysate and glucose.

Further preferred embodiments of the invention are characterized in that the cosmetically or dermatologically acceptable carrier is present as a powder, in stick form, as an aerosol spray, pump spray, liquid or gel roll-on product, cream, lotion, solution, gel or applied onto a substrate.

Deodorant or antiperspirant sticks may be present in gelled form, in an anhydrous base, based on a W/O emulsion, based on an O/W emulsion, based on a water-oil multiple emulsion, based on a nanoemulsion, and based on a microemulsion, wherein the oil phase may contain at least one silicone component or consists of at least one silicone component. The compositions according to the invention which are formulated as deodorant or antiperspirant sticks may be present in an anhydrous fatty base, based on a polyol-in-oil emulsion, based on an oil-in-polyol emulsion, based on a polyol-oil multiple emulsion, based on a nanoemulsion, and based on a microemulsion, wherein the polyol phase may be anhydrous or comprise only a small water content (for example 0.5-3 wt. %, relative to the total composition). Gel sticks may be formulated on the basis of fatty acid soaps, alditols, in particular dibenzylidene sorbitol, N-acylamino acid amides, 12-hydroxystearic acid, polyamides, polyamide derivatives, polysaccharides such as xanthan, polyglucomannans, guar, konjac, celluloses or starches, polyacrylates, polyacrylate derivatives and other gel formers.

Aerosol sprays, pump sprays, roll-on products and creams may be present as a water-in-oil emulsion, water-in-silicone oil emulsions, oil-in-water emulsion, silicone oil-in-water emulsion, water-in-oil microemulsion, oil-in-water microemulsion, silicone oil-in-water microemulsion, polyol-in-oil emulsion, oil-in-polyol emulsion, polyol-oil multiple emulsion, anhydrous suspension, preferably an anhydrous, oil-based suspension, alcoholic solution, in particular ethanolic solution, hydroalcoholic solution, in particular solutions with more than 50 wt. % of a water-ethanol mixture, glycolic solution, in particular as a solution in propylene glycol, glycerol, dipropylene glycol and polyethylene glycols which are liquid (under standard conditions), hydroglycolic solution, polyol solution, water-polyol solution, aqueous gel, lipogel and as an oil. All the stated compositions may be thickened, for example on the basis of fatty acid soaps, dibenzylidene sorbitol, N-acylamino acid amides, 12-hydroxystearic acid, polyacrylates of the carbomer and carbopol types, polyacrylamides and polysaccharides, which may be chemically and/or physically modified. The compositions may be transparent, translucent or opaque.

If the compositions according to the invention are present in the form of a stick, they preferably contain a wax matrix comprising at least one wax component with a melting point of >50° C.

Waxes are generally of a solid to brittle hard consistency, coarsely to finely crystalline, translucent to opaque, but not vitreous, and melt above 50° C. without decomposition. They are of low viscosity at just slightly above their melting point and exhibit strongly temperature-dependent consistency and solubility.

Natural vegetable waxes are, for example, preferred according to the invention, for example candelilla wax, carnauba wax, Japan wax, sugar cane wax, ouricoury wax, cork wax, sunflower wax, fruit waxes such as orange waxes, lemon waxes, grapefruit wax, and animal waxes, for example beeswax, shellac wax and spermaceti. It may be particularly preferred for the purposes of the invention to use hydrogenated or hardened waxes. Chemically modified waxes, in particular hard waxes, such as for example montan ester waxes, hydrogenated jojoba waxes and sasol waxes, may also be used as the wax component. Synthetic waxes which are likewise preferred according to the invention include for example polyalkylene waxes and polyethylene glycol waxes, C₂₀-C₄₀ dialkyl esters of dimer acids, C₃₀₋₅₀ alkyl beeswax and alkyl and alkylaryl esters of dimer fatty acids.

One particularly preferred wax component is selected from at least one ester of a saturated, monovalent C₁₆-C₆₀ alcohol and a saturated C₈-C₃₆ monocarboxylic acid. According to the invention, these also include lactides, the cyclic double esters of α-hydroxycarboxylic acids of the corresponding chain length. Esters of fatty acids and long-chain alcohols have proved particularly advantageous for the composition according to the invention because they impart excellent organoleptic properties to the antiperspirant preparation and elevated stability to the sticks. The esters are composed of saturated branched or unbranched monocarboxylic acids and saturated branched or unbranched monovalent alcohols. Esters of aromatic carboxylic acids or hydroxycarboxylic acids (for example 12-hydroxystearic acid) and saturated branched or unbranched alcohols may also be used according to the invention, providing that the wax component has a melting point of >50° C. It is particularly preferred to select the wax components from the group of esters of saturated branched or unbranched alkanecarboxylic acids of a chain length of 12 to 24 C atoms and saturated branched or unbranched alcohols of a chain length of 16 to 50 C atoms which have a melting point of >50° C.

In particular, C₁₆₋₃₆ alkyl stearates and C₁₈₋₃₈ alkylhydroxystearoyl stearates, C₂₀₋₄₀ alkyl erucates and cetearyl behenate may be advantageous as the wax component. The wax or wax components have a melting point of >50° C., preferably of >60° C.

A particularly preferred embodiment in stick form according to the invention contains a C₂₀-C₄₀ alkyl stearate as the wax component. This ester is known under the name Kester Wax® K82H or Kester Wax® K80H and is distributed by Koster Keunen Inc. This is a synthetic imitation of the monoester fraction of beeswax and is distinguished by its hardness, oil gelling capabilities and compatibility with a wide range of lipid components. This wax may be used as a stabilizer and consistency regulator for W/O and O/W emulsions. Kester Wax offers the advantage that it exhibits excellent oil gelling capabilities even at low concentrations and so does not make the stick preparation too heavy and allows it to be rubbed on extremely smoothly. A further particularly preferred embodiment according to the invention in stick form contains cetearyl behenate, i.e. mixtures of cetyl behenate and stearyl behenate, as the wax component. This ester is known under the name Kester Wax® K62 and is distributed by Koster Keunen Inc.

Further preferred wax components with a melting point of >50° C. are the triglycerides of saturated and optionally hydroxylated C₁₂₋₃₀ fatty acids, such as hardened triglyceride fats (hydrogenated palm oil, hydrogenated coconut oil, hydrogenated castor oil), glyceryl tribehenate (tribehenin) or glyceryl 12-hydroxystearate, furthermore synthetic full esters of fatty acids and glycols or polyols with 2-6 carbon atoms, providing they have a melting point of above 50° C., for example preferably C₁₈-C₃₆ acid triglycerides (Syncrowax® HGL-C). Hydrogenated castor oil, obtainable for example as the commercial product Cutina® HR, is particularly preferred according to the invention as the wax component.

Further preferred wax components with a melting point of >50° C. are saturated linear C₁₄-C₃₆ carboxylic acids, in particular myristic acid, palmitic acid, stearic acid and behenic acid and mixtures of these compounds, for example Syncrowax® AW 1C (C₁₈-C₃₆ fatty acids) or Cutina® FS 45 (palmitic and stearic acid).

Preferred deodorant or antiperspirant compositions according to the invention, in particular sticks, are characterized in that the wax component is selected from esters of a saturated, monovalent C₁₆-C₆₀ alkanol and a saturated C₈-C₃₆ monocarboxylic acid, in particular cetyl behenate, stearyl behenate and C₂₀-C₄₀ alkyl stearate, glycerol triesters of saturated linear C₁₂-C₃₀ carboxylic acids, which may be hydroxylated, candelilla wax, carnauba wax, beeswax, saturated linear C₁₄-C₃₆ carboxylic acids and mixtures of the above-stated substances. Particularly preferred wax component mixtures are selected from mixtures of cetyl behenate, stearyl behenate, hardened castor oil, palmitic acid and stearic acid. Further particularly preferred wax component mixtures are selected from mixtures of C₂₀-C₄₀ alkyl stearate, hardened castor oil, palmitic acid and stearic acid.

Preferred deodorant or antiperspirant sticks according to the invention based on an oil-in-water emulsion are characterized in that the wax component(s) is/are contained in total quantities of 4-20 wt. %, preferably of 8-15 wt. %, relative to the total composition. In a particularly preferred embodiment, the ester(s) of a saturated, monovalent C₁₆-C₆₀ alcohol and a saturated C₈-C₃₆ monocarboxylic acid, which constitute(s) the wax component(s), is/are contained in total quantities of 2-10 wt. %, preferably of 2-6 wt. %, relative to the total composition.

Compositions according to the invention which are formulated as an emulsion, in particular as an oil-in-water emulsion or polyol-in-water emulsion, preferably contain at least one nonionic oil-in-water emulsifier with an HLB value of greater than 7. These comprise emulsifiers which are generally known to a person skilled in the art, as are listed for example in Kirk-Othmer, “Encyclopedia of Chemical Technology”, 3rd edition, 1979, volume 8, page 913-916. For ethoxylated products, the HLB value is calculated according to the formula HLB=(100-L):5, L being the proportion by weight of lipophilic groups, i.e. the fatty alcohol or fatty acyl groups, in the ethylene oxide addition products, expressed in weight percent.

When selecting nonionic oil-in-water emulsifiers which are suitable according to the invention, it is particularly preferred to use a mixture of nonionic oil-in-water emulsifiers in order to be able to ensure optimum stability of the O/W emulsion compositions according to the invention. The individual emulsifier components here provide a proportion of the total HLB value or average HLB value of the oil-in-water emulsifier mixture in accordance with their proportion in the total quantity of the oil-in-water emulsifiers. According to the invention, the average HLB value of the oil-in-water emulsifier mixture amounts to 10-19, preferably 12-18 and particularly preferably 14-17. Such average HLB values are achieved by preferably combining oil-in-water emulsifiers from the HLB value ranges of 10-14, 14-16 and optionally 16-19 with one another. The oil-in-water emulsifier mixtures may of course also contain nonionic emulsifiers with HLB values in the range from >7-10 and 19-20; such emulsifier mixtures may likewise be preferred according to the invention. The deodorant or antiperspirant compositions according to the invention may, however, in another preferred embodiment also contain just one oil-in-water emulsifier with an HLB value in the range from 10-19.

Preferred deodorant or antiperspirant compositions according to the invention are characterized in that the nonionic oil-in-water emulsifiers are selected from ethoxylated C₈-C₂₄ alkanols with on average 10-100 mol of ethylene oxide per mol, ethoxylated C₈-C₂₄ carboxylic acids with on average 10-100 mol ethylene oxide per mol, silicone copolyols with ethylene oxide units or with ethylene oxide and propylene oxide units, alkyl mono- and oligoglycosides with 8 to 22 carbon atoms in the alkyl residue and the ethoxylated analogs thereof, ethoxylated sterols, partial esters of polyglycerols with 2 to 10 glycerol units and esterified with 1 to 4 saturated or unsaturated, linear or branched, optionally hydroxylated C₈-C₃₀ fatty acid residues, providing they have an HLB value of more than 7, and mixtures of the above-stated substances. The ethoxylated C₈-C₂₄ alkanols have the formula R¹O(CH₂CH₂O)ₙH, wherein R¹ denotes a linear or branched alkyl and/or alkenyl residue with 8-24 carbon atoms and n the average number of ethylene oxide units per molecule, for numbers from 10-100, preferably 10-30 mol of ethylene oxide on 1 mol of caprylic alcohol, 2-ethylhexyl alcohol, capric alcohol, lauryl alcohol, isotridecyl alcohol, myristyl alcohol, cetyl alcohol, isocetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and the technical mixtures thereof. Addition products of 10-100 mol of ethylene oxide onto technical fatty alcohols with 12-18 carbon atoms, such as for example coconut, palm, palm kernel or tallow fatty alcohol, are also suitable.

The ethoxylated C₈-C₂₄ carboxylic acids have the formula R¹(OCH₂CH₂)ₙOH, wherein R¹ denotes a linear or branched saturated or unsaturated acyl residue with 8-24 carbon atoms and n the average number of ethylene oxide units per molecule, for numbers from 10-100, preferably 10-30 mol of ethylene oxide onto 1 mol of caprylic acid, 2-ethylhexanoic acid, capric acid, lauric acid, isotridecanoic acid, myristic acid, cetylic acid, palmitoleic acid, stearic acid, isostearic acid, oleic acid, elaidic acid, petroselinic acid, arachidic acid, gadoleic acid, behenic acid, erucic acid and brassidic acid and the technical mixtures thereof. Addition products of 10-100 mol of ethylene oxide onto technical fatty acids with 12-18 carbon atoms, such as coconut, palm, palm kernel or tallow fatty acid, are also suitable. PEG-50 monostearate, PEG-100 monostearate, PEG-50 monooleate, PEG-100 monooleate, PEG-50 monolaurate and PEG-100 monolaurate are particularly preferred.

C₁₂-C₁₈ alkanols or C₁₂-C₁₈ carboxylic acids with in each case 10-30 units of ethylene oxide per molecule and mixtures of these substances are particularly preferably used, in particular ceteth-12, ceteth-20, ceteth-30, isoceteth-20, steareth-12, steareth-20, steareth-30, ceteareth-12, ceteareth-20, ceteareth-30, laureth-12 and beheneth-20.

C₈-C₂₂ alkyl mono- and oligoglycosides are furthermore preferably used. C₈-C₂₂ alkyl mono- and oligoglycosides are known, conventional commercial surfactants and emulsifiers. They are in particular produced by reacting glucose or oligosaccharides with primary alcohols having 8-22 carbon atoms. With regard to the glycoside residue, both monoglycosides, in which a cyclic sugar residue is glycosidically bound to the fatty alcohol, and oligomeric glycosides with a degree of oligomerization of up to approximately 8, preferably of 1-2, are suitable. The degree of oligomerization is here a statistical mean which is based on a homologue distribution conventional for such technical products. Products, which are obtainable under the trademark Plantacare®, contain a glycosidically bound C₈-C₁₆ alkyl group on an oligoglucoside residue, the average degree of oligomerization of which is at 1-2, in particular 1.1-1.4. Particularly preferred C₈-C₂₂ alkyl mono- and oligoglycosides are selected from octyl glucoside, decyl glucoside, lauryl glucoside, palmityl glucoside, isostearyl glucoside, stearyl glucoside, arachidyl glucoside and behenyl glucoside and mixtures thereof. The acyl glucamides derived from glucamine are also suitable as nonionic oil-in-water emulsifiers.

Ethoxylated sterols, in particular ethoxylated soy sterols, are also oil-in-water emulsifiers which are suitable according to the invention. The degree of ethoxylation must be greater than 5, preferably at least 10 if an HLB value of above 7 is to be exhibited. Suitable commercial products are for example PEG-10 soy sterol, PEG-16 soy sterol and PEG-25 soy sterol.

Partial esters of polyglycerols with 2 to 10 glycerol units and esterified with 1 to 4 saturated or unsaturated, linear or branched, optionally hydroxylated C₈-C₃₀ fatty acid residues, are furthermore preferably used, provided they exhibit an HLB value of greater than 7. Particular preference is given to diglycerol monocaprylate, diglycerol monocaprate, diglycerol monolaurate, triglycerol monocaprylate, triglycerol monocaprate, triglycerol monolaurate, tetraglycerol monocaprylate, tetraglycerol monocaprate, tetraglycerol monolaurate, pentaglycerol monocaprylate, pentaglycerol monocaprate, pentaglycerol monolaurate, hexaglycerol monocaprylate, hexaglycerol monocaprate, hexaglycerol monolaurate, hexaglycerol monomyristate, hexaglycerol monostearate, decaglycerol monocaprylate, decaglycerol monocaprate, decaglycerol monolaurate, decaglycerol monomyristate, decaglycerol monoisostearate, decaglycerol monostearate, decaglycerol monooleate, decaglycerol monohydroxystearate, decaglycerol dicaprylate, decaglycerol dicaprate, decaglycerol dilaurate, decaglycerol dimyristate, decaglycerol diisostearate, decaglycerol distearate, decaglycerol dioleate, decaglycerol dihydroxystearate, decaglycerol tricaprylate, decaglycerol tricaprate, decaglycerol trilaurate, decaglycerol trimyristate, decaglycerol triisostearate, decaglycerol tristearate, decaglycerol trioleate and decaglycerol trihydroxystearate.

Particularly preferred deodorant or antiperspirant compositions according to the invention are characterized in that the nonionic oil-in-water emulsifier is contained in a total quantity of 0.5-10 wt. %, particularly preferably of 1-4 wt. % and extremely preferably of 1.5-3 wt. %, relative to the total composition.

Preferred compositions according to the invention which are formulated as an emulsion or stick preferably furthermore contain at least one nonionic water-in-oil emulsifier with an HLB value of greater than 1.0 and less than or equal to 7.0, selected from the mono- and diesters of ethylene glycol and the mono-, di-, tri- and tetraesters of pentaerythritol with linear saturated fatty acids having 12-30, in particular 14-22 carbon atoms, which may be hydroxylated, and mixtures thereof, as consistency providers, and/or water-binding agents. Mono- and diesters are preferred according to the invention.

C₁₂-C₃₀ fatty acid residues which are preferred according to the invention are selected from lauric acid, myristic acid, palmitic acid, stearic acid, arachidic acid and behenic acid residues; the stearic acid residue is particularly preferred. Nonionic water-in-oil emulsifiers with an HLB value of greater than 1.0 and less than or equal to 7.0 which are particularly preferred according to the invention are selected from pentaerythrityl monostearate, pentaerythrityl distearate, pentaerythrityl tristearate, pentaerythrityl tetrastearate, ethylene glycol monostearate, ethylene glycol distearate and mixtures thereof. Water-in-oil-emulsifiers with an HLB value of greater than 1.0 and less than or equal to 7.0 which are particularly preferred according to the invention are for example obtainable as commercial products Cutina® PES (INCI: pentaerythrityl distearate), Cutina® AGS (INCI: glycol distearate) or Cutina® EGMS (INCI: glycol stearate). These commercial products are themselves already mixtures of mono- and diesters (tri- and tetraesters are also present in the pentaerythrityl esters). It may be preferred according to the invention to use only one single water-in-oil emulsifier. In another preferred embodiment, the compositions according to the invention contain mixtures, in particular technical mixtures, of at least two water-in-oil emulsifiers. A technical mixture is for example taken to mean a commercial product such as Cutina® PES.

Apart from the stated water-in-oil emulsifiers based on ethylene glycol or pentaerythrityl esters, in one preferred embodiment at least one further nonionic water-in-oil emulsifier with an HLB value of greater than 1.0 and less than or equal to 7.0 may be contained, the proportion of which in the total weight of nonionic water-in-oil emulsifiers with an HLB value of greater than 1.0 and less than or equal to 7.0 should, however, preferably be no greater than 80%. In a particularly preferred embodiment, the compositions according to the invention only contain the at least one additional water-in-oil emulsifier with an HLB value of greater than 1.0 and less than or equal to 7.0 in a proportion by weight of at most 10% or are free of additional water-in-oil emulsifiers. Some of these additional suitable emulsifiers are listed, for example, in Kirk-Othmer, “Encyclopedia of Chemical Technology”, 3rd edition, 1979, volume 8, page 913. The HLB value for ethoxylated addition products may also be calculated as already mentioned.

Preferably suitable water-in-oil emulsifiers are:
linear saturated alkanols with 12-30 carbon atoms, in particular with 16-22 carbon atoms, in particular cetyl alcohol, stearyl alcohol, arachidyl alcohol, behenyl alcohol and lanolin alcohol or mixtures of these alcohols, as they are obtainable from the industrial hydrogenation of plant and animal fatty acids,
esters and in particular partial esters of a polyol with 3-6 C atoms and linear saturated and unsaturated fatty acids with 12-30, in particular 14-22 C atoms, which may be hydroxylated. Such esters or partial esters are for example the mono- and diesters of glycerol or the monoesters of propylene glycol with linear saturated and unsaturated C₁₂-C₃₀ carboxylic acids, which may be hydroxylated, in particular those with palmitic and stearic acid, sorbitan mono-, di- or triesters of linear saturated and unsaturated C₁₂-C₃₀ carboxylic acids, which may be hydroxylated, in particular those of myristic acid, palmitic acid, stearic acid or of mixtures of these fatty acids and the methylglucose mono- and diesters of linear saturated and unsaturated C₁₂-C₃₀ carboxylic acids, which may be hydroxylated;
sterols, thus steroids which bear a hydroxyl group on the C₃ atom of the steroid skeleton and may be isolated both from animal tissue (zoosterols, for example cholesterol, lanosterol) and from plants (phytosterols, for example ergosterol, stigmasterol, sitosterol) and from fungi and yeast (mycosterols) and may be slightly ethoxylated (1-5 EO);
alkanols and carboxylic acids with in each case 8-24 C atoms, in particular with 16-22 C atoms, in the alkyl group and 1-4 ethylene oxide units per molecule which have an HLB value of greater than 1.0 and less than or equal to 7.0,
glycerol monoethers of saturated and/or unsaturated, branched and/or unbranched alcohols of a chain length of 8-30, in particular 12-18, carbon atoms.
partial esters of polyglycerols with n=2 to 10 glycerol units and esterified with 1 to 5 saturated or unsaturated, linear or branched, optionally hydroxylated C₈-C₃₀ fatty acid residues, providing they have an HLB value of less than or equal to 7,
and mixtures of the above-stated substances.

It may be preferred according to the invention to use only one single additional water-in-oil emulsifier. In another preferred embodiment, the compositions according to the invention contain mixtures, in particular technical mixtures, of at least two additional water-in-oil emulsifiers. A technical mixture is for example taken to mean a commercial product such as Cutina® GMS which is a mixture of glyceryl monostearate and glyceryl distearate.

Additional water-in-oil emulsifiers which may particularly advantageously be used are stearyl alcohol, cetyl alcohol, glyceryl monostearate, in particular in the form of the commercial products Cutina® GMS and Cutina® MD (from Cognis), glyceryl distearate, glyceryl monocaprate, glyceryl monocaprylate, glyceryl monolaurate, glyceryl monomyristate, glyceryl monopalmitate, glyceryl monohydroxystearate, glyceryl monooleate, glyceryl monolanolate, glyceryl dimyristate, glyceryl dipalmitate, glyceryl dioleate, propylene glycol monostearate, propylene glycol monolaurate, sorbitan monocaprylate, sorbitan monolaurate, sorbitan monomyristate, sorbitan monopalmitate, sorbitan monostearate, sorbitan sesquistearate, sorbitan distearate, sorbitan dioleate, sorbitan sesquioleate, sucrose distearate, arachidyl alcohol, behenyl alcohol, polyethylene glycol (2) stearyl ether (steareth-2), steareth-5, oleth-2, diglycerol monostearate, diglycerol monoisostearate, diglycerol monooleate, diglycerol dihydroxystearate, diglycerol distearate, diglycerol dioleate, triglycerol distearate, tetraglycerol monostearate, tetraglycerol distearate, tetraglycerol tristearate, decaglycerol pentastearate, decaglycerol pentahydroxystearate, decaglycerol pentaisostearate, decaglycerol pentaoleate, soy sterol, PEG-1 soy sterol, PEG-5 soy sterol, PEG-2 monolaurate and PEG-2 monostearate.

Particularly preferred deodorant or antiperspirant compositions according to the invention are characterized in that at least one water-in-oil emulsifier is contained in a total quantity of 0.1-15 wt. %, preferably of 0.5-8.0 wt. % and particularly preferably of 1-4 wt. %, relative to the total composition. Quantities of 2-3-3.5 wt. %, relative to the total weight of the composition, may furthermore be extremely preferred according to the invention.

Further W/O emulsifiers which are preferred according to the invention are silicone-free polymeric water-in-oil emulsifiers, in particular PEG-30 dipolyhydroxystearate, obtainable for example under the trade name Arlacel P 135 from Uniqema. Low-viscosity and even sprayable water-in-oil emulsions can be formulated in particular with the assistance of this emulsifier.

Particularly preferred compositions according to the invention which are formulated as a water-in-oil emulsion preferably furthermore contain at least one silicone-based water-in-oil emulsifier. The at least one silicone-based water-in-oil emulsifier is preferably contained in a quantity of 0.5-5 wt. %, particularly preferably of 1.0-1.5-2.5-3-3.5 wt. %, in each case relative to the total weight of the emulsion.

One group of silicone-based water-in-oil emulsifiers which is particularly preferred according to the invention are poly-(C₂-C₃)alkylene glycol-modified silicones, the former INCI name of which was dimethicone copolyol, with the current INCI name PEG-x dimethicone (with x=2-20, preferably 3-17, particularly preferably 11-12), bis-PEG-y dimethicone (with y=3-25, preferably 4-20), PEG/PPG-a/b dimethicone (wherein a and b mutually independently denote numbers from 2-30, preferably 3-30 and particularly preferably 12-20, in particular 14-18), bis-PEG/PPG-c/d dimethicone (wherein c and d mutually independently denote numbers from 10-25, preferably 14-20 and particularly preferably 14-16) and bis-PEG/PPG-e/f PEG/PPG-g/h dimethicone (wherein e, f, g and h mutually independently denote numbers from 10-20, preferably 14-18 and particularly preferably 16). Particularly preferred substances are PEG/PPG-18/18 dimethicone, which is commercially available in a 1:9 mixture with cyclomethicone as DC 3225 C or DC 5225 C, PEG/PPG-4/12 dimethicone, which is obtainable under the name Abil B 8852, and bis-PEG/PPG-14/14 dimethicone, which is commercially available in a mixture with cyclomethicone as Abil EM 97 (Goldschmidt), bis-PEG/PPG-20/20 dimethicone, which is obtainable under the name Abil B 8832, PEG/PPG-5/3 trisiloxane (Silsoft 305), and PEG/PPG-20/23 dimethicone (Silsoft 430 and Silsoft 440).

Further silicone-based W/O emulsifiers which are preferred according to the invention are poly-(C₂-C₃)-alkylene glycol-modified silicones, which are hydrophobically modified with C₄-C₁₈ alkyl groups, particularly preferably cetyl PEG/PPG-10/1 dimethicone (formerly: cetyl dimethicone copolyol, obtainable as Abil EM 90 or in a mixture of polyglycerol 4-isostearate, cetyl PEG/PPG-10/1 dimethicone and hexyl laurate under the tradename Abil WE 09), furthermore alkyl methicone copolyols and alkyl dimethicone ethoxy glucoside.

The following table is a compilation of various oil-in-water emulsifiers and water-in-oil emulsifiers and their HLB values. The HLB values may, however, also be calculated according to Griffin, as is for example explained and tabulated in ROMPP Chemie Lexikon, in particular in the online version of November 2003, and the handbooks by Fiedler, Kirk-Othmer and Janistyn cited therein under the headword “HLB system”. Where the literature states differing details relating to a substance's HLB value, the HLB value used for the teaching according to the invention should be that which is closest to the value calculated according to Griffin. Should it not be possible to determine an unambiguous HLB value in this manner, the HLB value stated by the manufacturer of the emulsifier should be used for the teaching according to the invention. Should this too not be possible, the HLB value should be determined experimentally.

### HLB Value Chemical Name

(from H. Janistyn, Handbuch der Kosmetika und Riechstoffe, Hüthig-Verlag Heidelberg, 3rd edition, 1978, volume 1, page 470 and volume 3, pages 68-78))
1 triglycerides of saturated fatty acids glyceryl trioleate
1.5 ethylene glycol distearate
1.6 Pur-Cellin oil
1.8 sorbitan trioleate
   glycerol diacetate
2.1 sorbitan tristearate
2.4 propylene glycol lactostearate
2.7 glycerol monooleate
   sorbitol dioleate
2.8 glycerol monostearate
   propylene glycol mono-/distearate, not self-emulsifying
2.9 ethylene glycol monostearate
3.0 decaglycerol decaoleate
   decaglycerol decastearate
   Generol 122 (rapeseed sterols)
   sucrose distearate
3.1 decaglycerol decaoleate
   glyceryl monoricinoleate
   pentaerythrityl monostearate
   pentaerythrityl sesquioleate
3.2 ethylene glycol monodistearate, not self-emulsifying
   glycol stearate
3.3 glycerol monolaurate
3.4 propylene glycol monostearate
3.5 ethylene glycol monostearate
   pentaerythrityl monooleate
   polyethylene glycol (100) monooleate
3.6 glycerol mono-/dioleate, not self-emulsifying
   monoethoxylauryl ether
3.7 sorbitan sesquioleate, (Dehymuls SSO)
3.8 glycerol monodistearate, not self-emulsifying
   polyethylene glycol (100) monostearate,
   diglycerol sesquioleate
   N,N-dimethylcaproamide
   pentaerythritol monotallate
   propylene glycol monolaurate
4.0 decaglycerol octaoleate
4.3 sorbitan monooleate (Dehymuls SMO)
   diethylene glycol monostearate
4.4 1,2-propylene glycol monodistearate, self-emulsifying
4.5 glycerol monostearate-palmitate (90%), not self-emulsifying
   propylene glycol monolaurate
4.7 sorbitan monostearate (Dehymuls SMS)
   diethylene glycol monooleate
4.8 pentaerythritol monolaurate
4.9 polyoxyethylene (2) oleyl alcohol (polyoxyethylene (2) oleyl ether)
   polyoxyethylene (2) stearyl alcohol (polyoxyethylene (2) stearyl ether)
5.0 ethylene glycol monodistearate
   Generol 122 E 5 (PEG-5 soy sterol)
   polyethylene glycol (100) monoricinoleate
   polyethylene glycol (200) distearate
   polyglyceryl 3-isostearate (for example Isolan GI 34 from Tego)
5.9 polyethylene glycol (200) dilaurate
6.0 decaglycerol tetraoleate
   polyethylene glycol (100) monolaurate
   polyethylene glycol (200) dioleate
6.1 diethylene glycol monolaurate (diglycol laurate)
6.3 polyethylene glycol (300) dilaurate
6.4 glycerol monoricinoleate
   glycerol sorbitan monolaurate
6.5 diethylene glycol monolaurate
   sodium stearoyl 2-lactylate
6.7 sorbitan monopalmitate
6.8 glycerol monococoate
   glycerol monolaurate
7.0 polyoxyethylene (2) C₁₀-C₁₄ fatty alcohol ether, laureth-2 (Dehydrol LS 2)
   sucrose distearate
7.2 polyethylene glycol (400) dioleate
   sucrose dioleate
7.4 polyethylene glycol (100) monolaurate
   sucrose dipalmitate
7.5 sucrose dipalmitate
7.6 glycerol sorbitan laurate
7.8 polyethylene glycol (400) distearate
7.9 polyethylene glycol (200) monostearate
   polyoxyethylene (3) tridecyl alcohol
8-8.2 polyethylene glycol (400) distearate
8.0 polyoxyethylene (3) C₁₀-C₁₄ fatty alcohol ether, laureth-3 (Dehydrol LS 3)
   N,N-dimethyllauramide
   sodium lauroyl lactylate, sodium lauroyl 2-lactylate
   polyethylene glycol (200) monooleate
   polyethylene glycol (220) monotallate
   polyethylene glycol (1500) dioleate
   polyoxyethylene (4) oleyl alcohol
   polyoxyethylene (4) stearylcetyl ether
8.2 triglycerol monooleate
8.3 diethylene glycol monolaurate
8.4 polyoxyethylene (4) cetyl ether
   polyoxyethylene glycol (400) dioleate
8.5 sodium caproyl lactylate
   polyethylene glycol (200) monostearate
   sorbitan monooleate
8.6 sorbitan monolaurate (Dehymuls SML)
   polyethylene glycol (200) monolaurate
8.8 polyoxyethylene (4) myristyl ether
   polyethylene glycol (400) dioleate
8.9 nonylphenol, polyethoxylated with 4 mol EO
9.0 oleth-5 (for example Eumulgin O 5)
9.2-9.7 polyoxyethylene (4) lauryl alcohol (depending on commercial product, for example Brij 30, Dehydrol LS 4)
9.3 polyoxyethylene (4) tridecyl alcohol
9.6 polyoxyethylene (4) sorbitan monostearate
9.8 polyethylene glycol (200) monolaurate
10-11 polyethylene glycol (400) monooleate
10.0 didodecyldimethylammonium chloride
10.0 polyethylene glycol (200) monolaurate
   polyethylene glycol (400) dilaurate
   polyethylene glycol (600) dioleate
   polyoxyethylene (4) sorbitan monostearate
   polyoxyethylene (5) sorbitan monooleate
10.2 polyoxyethylene (40) sorbitol hexaoleate
10.4-10.6 polyoxyethylene glycol (600) distearate
10.5 polyoxyethylene (20) sorbitan tristearate
10.6 sucrose monostearate
10.7 sucrose monooleate
11-11.4 polyethylene glycol (400) monooleate
11.0 polyethylene glycol (350) monostearate
   polyethylene glycol (400) monotallate
   polyoxyethylene glycol (7) monostearate
   polyoxyethylene glycol (8) monooleate
   polyoxyethylene (20) sorbitan trioleate
   polyoxyethylene (6) tridecyl alcohol
11.1 polyethylene glycol (400) monostearate
11.2 polyoxyethylene (9) monostearate
   sucrose monooleate
   sucrose monostearate
11.4 polyoxyethylene (50) sorbitol hexaoleate
   sucrose monotallate
   sucrose stearate-palmitate
11.6 polyoxyethylene glycol (400) monoricinoleate
11.7 sucrose monomyristate
   sucrose monopalmitate
12.0 PEG-10 soy sterol (for example Generol 122 E 10)
   triethanolamine oleate
12.2-12.3 nonylphenol, ethoxylated with 8 mol EO
12.2 sucrose monomyristate
12.4 sucrose monolaurate
   polyoxyethylene (10) oleyl alcohol,
   polyoxyethylene (10) oleyl ether
   polyoxyethylene (10) stearyl alcohol,
   polyoxyethylene (10) stearyl ether
12.5 polyoxyethylene (10) stearylcetyl ether
12.7 polyoxyethylene (8) tridecyl alcohol
12.8 polyoxyethylene glycol (400) monolaurate
   sucrose monococoate
12.9 polyoxyethylene (10) cetyl ether
13 glycerol monostearate, ethoxylated (20 mol EO)
13.0 Eumulgin O 10
   Eumulgin 286
   Eumulgin B1 (ceteareth-12)
13.0 C12 fatty amines, ethoxylated (5 mol EO)
13.1 nonylphenol, ethoxylated (9.5 mol EO)
13.2 polyethylene glycol (600) monostearate
   polyoxyethylene (16) tall oil
13.3 polyoxyethylene (4) sorbitan monolaurate
13.5 nonylphenol, ethoxylated (10.5 mol EO)
   polyethylene glycol (600) monooleate
13.7 polyoxyethylene (10) tridecyl alcohol
   polyethylene glycol (660) monotallate
   polyethylene glycol (1500) monostearate,
   polyoxyethylene glycol (1500) dioleate
13.9 polyethylene glycol (400) monococoate
   polyoxyethylene (9) monolaurate
14-16 Eumulgin HRE 40 (castor oil, ethoxylated with 40 EO and hydrogenated)
14.0 polyoxyethylene (12) lauryl ether
   polyoxyethylene (12) tridecyl alcohol
14.2 polyoxyethylene (15) stearyl alcohol
14.3 polyoxyethylene (15) stearylcetyl ether
14.4 mixture of C12-C15 fatty alcohols with 12 mol EO
14.5 polyoxyethylene (12) lauryl alcohol
14.8 polyoxyethylene glycol (600) monolaurate
14.9-15.2 sorbitan monostearate, ethoxylated with 20 EO (for example Eumulgin SMS 20)
15-15.9 sorbitan monooleate, ethoxylated with 20 EO (for example Eumulgin SMO 20)
15.0 PEG-20 glyceryl stearate (for example Cutina E 24)
   PEG-40 castor oil (for example Eumulgin RO 40) decyl glucoside (Oramix NS 10)
   dodecyl glucoside (Plantaren APG 600)
   dodecyltrimethylammonium chloride
   nonylphenol, ethoxylated with 15 mol EO
   polyethylene glycol (1000) monostearate,
   polyoxyethylene (600) monooleate
15-17 Eumulgin HRE 60 (castor oil, ethoxylated with 60 EO and hydrogenated)
15.3 C₁₂ fatty amines, polyethoxylated with 12 mol EO polyoxyethylene (20) oleyl alcohol, polyoxyethylene (20) oleyl ether
15.4 polyoxyethylene (20) stearyl cetyl ether (for example Eumulgin B 2 (ceteareth-20))
15.5 polyoxyethylene (20) stearyl alcohol
15.6 polyoxyethylene glycol (1000) monostearate
   polyoxyethylene (20) sorbitan monopalmitate
15.7 polyoxyethylene (20) cetyl ether
15.9 disodium triethanolamine distearyl heptaglycol ether sulfosuccinate
16.0 nonylphenol, ethoxylated with 20 mol EO
   polyoxyethylene (25) propylene glycol stearate
16-16.8 polyoxyethylene (30) monostearate
16.3-16.9 polyoxyethylene (40) monostearate
16.5-16.7 polyoxyethylene (20) sorbitan monolaurate (for example Eumulgin SML 20)
16.6 polyoxyethylene (20) sorbitol
16.7 C₁₈ fatty amines, polyethoxylated with 5 mol EO
   polyoxyethylene (23) lauryl alcohol
17.0 ceteareth-30, for example Eumulgin B 3
   octyl glucoside (Triton CG 110)
   polyoxyethylene (30) glyceryl monolaurate
17.1 nonylphenol, ethoxylated with 30 mol EO
17.4 polyoxyethylene (40) stearyl alcohol

Further preferred compositions according to the invention are characterized in that the total content of nonionic and ionic emulsifiers and/or surfactants with an HLB value of above 8 amounts to at most 20 wt. %, preferably to at most 15 wt. %, particularly preferably to at most 10 wt. %, particularly preferably to at most 7 wt. %, furthermore particularly preferably to at most 4 wt. % and extremely preferably to at most 3 wt. %, in each case relative to the total composition according to the invention.

Preferred compositions according to the invention which are present as an emulsion, suspension or stick preferably furthermore contain at least one oil which is liquid at 20° C. which is not a fragrance component and not an essential oil. Oils which are preferred according to the invention are selected from branched saturated or unsaturated fatty alcohols with 6-30 carbon atoms. These alcohols are frequently also designated Guerbet alcohols, as they can be obtained by the Guerbet reaction. Preferred alcohol oils are hexyldecanol (Eutanol® G 16, Guerbitol® T 16), octyidodecanol (Eutanol®G, Guerbitol® 20), 2-ethylhexyl alcohol and the commercial products Guerbitol® 18, Isofol® 12, Isofol® 16, Isofol® 24, Isofol® 36, Isocarb® 12, Isocarb® 16 or Isocarb® 24. Further preferred oil components are mixtures of Guerbet alcohols and Guerbet alcohol esters, for example the commercial product Cetiol® PGL (hexyldecanol and hexyldecyl laurate).

Further oils which are preferred according to the invention are selected from the triglycerides of linear or branched, saturated or unsaturated, optionally hydroxylated C₈₋₃₀ fatty acids. It may be particularly suitable to use natural oils, for example soy oil, cottonseed oil, sunflower oil, palm oil, palm kernel oil, linseed oil, almond oil, castor oil, corn oil, olive oil, rapeseed oil, sesame oil, thistle oil, wheat germ oil, peach stone oil and the liquid fractions of coconut oil and the like. Synthetic triglyceride oils are, however, also suitable, in particular capric/caprylic triglycerides, for example the commercial products Myritol® 318, Myritol® 331 (Cognis) or Miglyol® 812 (Hüls) with unbranched fatty acid residues and glyceryl triisostearin and the commercial products Estol® GTEH 3609 (Uniqema) or Myritol® GTEH (Cognis) with branched fatty acid residues.

Further oils which are particularly preferred according to the invention are selected from the dicarboxylic acid esters of linear or branched C₂-C₁₀ alkanols, in particular diisopropyl adipate, di-n-butyl adipate, di-(2-ethylhexyl) adipate, dioctyl adipate, diethyl-/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Further oils which are particularly preferred according to the invention are selected from the addition products of 1 to 5 propylene oxide units onto mono- or polyhydric C₈₋₂₂ alkanols such as octanol, decanol, decanediol, lauryl alcohol, myristyl alcohol and stearyl alcohol, for example PPG-2 myristyl ether and PPG-3 myristyl ether (Witconol® APM).

Further oil components which are preferred according to the invention are selected from the esters of linear or branched saturated or unsaturated fatty alcohols having 2-30 carbon atoms with linear or branched saturated or unsaturated fatty acids having 2-30 carbon atoms, which may be hydroxylated. These include hexyldecyl stearate (Eutanol® G 16 S), hexyldecyl laurate, isodecyl neopentanoate, isononyl isononanoate, 2-ethylhexyl palmitate (Cegesoft® C 24) and 2-ethylhexyl stearate (Cetiol® 868). The following are likewise conditionally suitable: isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyloctanoate, diisotridecyl acetate, n-butyl stearate, n-hexyl laurate, n-decyl oleate, oleyl oleate, oleyl erucate, erucyl oleate, erucyl erucate, ethylene glycol dioleate and dipalmitate.

Further oil components which are preferred according to the invention are selected from the addition products of at least 6 ethylene oxide and/or propylene oxide units onto mono- or polyhydric C₃₋₂₂ alkanols such as butanol, butanediol, myristyl alcohol and stearyl alcohol, for example PPG-14 butyl ether (Ucon Fluid® AP), PPG-9 butyl ether (Breox® B25), PPG-10 butanediol (Macol® 57) and PPG-15 stearyl ether (Arlamol® E).

Further oil components which are preferred according to the invention are selected from the C₈-C₂₂ fatty alcohol esters of monobasic or polybasic C₂-C₇ hydroxycarboxylic acids, in particular the esters of glycolic acid, lactic acid, malic acid, tartaric acid, citric acid and salicylic acid. Such esters based on linear C_{14/15} alkanols, for example C₁₂-C₁₅ alkyl lactate, and of C_{12/13} alkanols branched in position 2 are obtainable under the trademark Cosmacol® from Nordmann, Rassmann GmbH & Co, Hamburg, in particular the commercial products Cosmacol® ESI, Cosmacol® EMI and Cosmacol® ETI.

Further oil components which are preferred according to the invention are selected from symmetrical, asymmetrical or cyclic esters of carbonic acid with fatty alcohols, for example glycerol carbonate, dicaprylyl carbonate (Cetiol® CC) or the esters of DE 197 56 454 A1.

Further oil components which are preferred according to the invention are selected from the esters of dimers of unsaturated C₁₂-C₂₂ fatty acids (dimer fatty acids) with monovalent linear, branched or cyclic C₂-C₁₈ alkanols or with polyhydric linear or branched C₂-C₆ alkanols. It may be extremely preferred according to the invention to use mixtures of the above-stated oils.

Further oil components which are preferred according to the invention are selected from silicone oils and hydrocarbon oils.

Silicone oils which are preferred according to the invention are selected from dialkyl- and alkylarylsiloxanes, among which are included for example not only dimethylpolysiloxane and methylphenylpolysiloxane, but also hexamethyldisiloxane, octamethyltrisiloxane and decamethyltetrasiloxane.

Further silicone oils which are preferred according to the invention are selected from volatile silicone oils, which may be cyclic, such as for example octamethylcyclotetrasiloxane, decamethylcyclopentasiloxane and dodecamethylcyclohexasiloxane and mixtures thereof, such as are for example contained in the commercial products DC 244, 245, 344 and 345 from Dow Corning, or linear, for example hexamethyldisiloxane (L₂), octamethyltrisiloxane (L₃), decamethyltetrasiloxane (L₄), any desired binary and ternary mixtures of L₂, L₃ and/or L₄, as are for example contained in the commercial products DC 2-1184, Dow Corning® 200 (0.65 cSt) and Dow Corning® 200 (1.5 cSt) from Dow Corning.

Further silicone oils which are preferred according to the invention are selected from non-volatile relatively high molecular weight linear dimethylpolysiloxanes, commercially available for example under the name Dow Corning® 190, Dow Corning® 200 Fluid with viscosities in the range from 5-100 cSt, preferably 5-50 cSt or also 5-10 cSt, and Baysilon® 350 M.

Natural and synthetic hydrocarbons which are preferred according to the invention are selected from paraffin oils, isohexadecane, isoeicosane, polyisobutenes and polydecenes, which are obtainable for example under the name Emery® 3004, 3006, 3010 or under the name Ethylflo® from Albemarle or Nexbase® 2004G from Nestle, and 1,3-di-(2-ethylhexyl)-cyclohexane (Cetiol®S). Particularly preferred deodorant or antiperspirant compositions according to the invention are characterized in that the oil(s) liquid at 20° C. is/are contained in a total quantity of 0.1-80 wt. %, preferably of 2-20 wt. %, particularly preferably of 3-15 wt. %, in each case relative to the total weight of the composition.

In a further preferred embodiment of the invention, a proportion of at least 80 wt. % of the oil components exhibits a refractive index n_{D} of 1.39-1.51. It is particularly preferred for 5-40-50 wt. %, extremely preferably 10-12-25-30 wt. % of the oil components to exhibit a refractive index n_{D} of 1.43-1.58, preferably of 1.44-1.51, particularly preferably of 1.45-1.47-1.49, at 20° C. (measured at λ=589 nm).

Compositions which are particularly preferred according to the invention furthermore preferably contain at least one skin-cooling active ingredient. Skin-cooling active ingredients which are suitable according to the invention are for example menthol, isopulegol and menthol derivatives, for example menthyl lactate, menthyl glycolate, menthyl pyrrolidone carboxylic acid, menthyl methyl ether, menthoxypropanediol, menthone glycerol acetal, (9-methyl-6-(1-methylethyl)-1,4-dioxaspiro(4.5)decane-2-methanol), monomenthyl succinate and 2-hydroxymethyl-3,5,5-trimethylcyclohexanol. Preferred skin-cooling active ingredients are menthol, isopulegol, menthyl lactate, menthoxypropanediol and menthyl pyrrolidone carboxylic acid and mixtures of these substances, in particular mixtures of menthol and menthyl lactate, menthol, menthol glycolate and menthyl lactate, menthol and menthoxypropanediol or menthol and isopulegol.

It is particularly preferred according to the invention for at least one skin-cooling active ingredient to be contained in a total quantity of 0.01-1 wt. %, particularly preferably of 0.02-0.5 wt. % and extremely preferably of 0.05-0.2-0.3 wt. %, in each case relative to the total weight of the composition.

Particularly preferred compositions according to the invention, which are formulated as a propellant gas driven aerosol, contain at least propellant. Preferred propellants (propellant gases) are propane, propene, n-butane, iso-butane, iso-butene, n-pentane, pentene, iso-pentane, iso-pentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane (INCI: hydrofluorocarbon 152a), and specifically both individually and in combination. Hydrophilic propellant gases, such as for example carbon dioxide, may advantageously be used for the purposes of the present invention if a small proportion of hydrophilic gases is selected and a lipophilic propellant gas (for example propane/butane) is present in excess. Propane, n-butane, iso-butane and mixtures of these propellant gases are particularly preferred. It has been found that it may be particularly preferred according to the invention to use n-butane as the sole propellant gas.

The quantity of propellant preferably amounts to 20-80-90 wt. %, particularly preferably to 30-70-75 wt. % and extremely preferably to 40-50 wt. %, in each case relative to the total weight of the preparation consisting of the composition according to the invention and the propellant.

Vessels of metal (aluminum, tin plate, tin), of protected or non-splintering plastics or of glass coated on the outside with plastics may be considered as the pressurized gas container, the selection of which is made on the basis not only of pressure resistance and breaking strength, corrosion resistance, ease of filling but also of aesthetic considerations, ease of handling, printability etc. Special internal protection lacquers ensure corrosion resistance relative to the composition according to the invention. The compositions according to the invention which are atomized with a propellant may be packaged either directly with the propellant, or alternatively in a multichamber container, in which the composition according to the invention is present physically separated from the propellant.

Preferred compositions according to the invention furthermore contain at least one water-soluble polyhydric C₂-C₈ alkanol with 2-6 hydroxyl groups and/or at least one water-soluble polyethylene glycol with 3-20 ethylene oxide units and mixtures thereof. These components are preferably selected from 1,2-propylene glycol, 2-methyl-1,3-propanediol, glycerol, butylene glycols such as 1,2-butylene glycol, 1,3-butylene glycol and 1,4-butylene glycol, pentylene glycols such as 1,2-pentanediol and 1,5-pentanediol, hexanediols such as 1,6-hexanediol, hexanetriols such as 1,2,6-hexanetriol, 1,2-octanediol, 1,8-octanediol, dipropylene glycol, tripropylene glycol, diglycerol, triglycerol, erythritol, sorbitol and mixtures of the above-stated substances. Suitable water-soluble polyethylene glycols are selected from PEG-3, PEG-4, PEG-6, PEG-7, PEG-8, PEG-9, PEG-10, PEG-12, PEG-14, PEG-16, PEG-18 and PEG-20 and mixtures thereof, with PEG-3 to PEG-8 being preferred. Preferred deodorant or antiperspirant compositions according to the invention are characterized in that the at least one water-soluble polyhydric C₂-C₈ alkanol with 2-6 hydroxyl groups and/or at least one water-soluble polyethylene glycol with 3-20 ethylene oxide units is selected from 1,2-propylene glycol, 2-methyl-1,3-propanediol, glycerol, butylene glycols such as 1,2-butylene glycol, 1,3-butylene glycol and 1,4-butylene glycol, pentylene glycols such as 1,2-pentanediol and 1,5-pentanediol, hexanediols such as 1,6-hexanediol, hexanetriols such as 1,2,6-hexanetriol, 1,2-octanediol, 1.8-octanediol, dipropylene glycol, tripropylene glycol, diglycerol, triglycerol, erythritol, sorbitol and mixtures of the above-stated substances.

Particularly preferred deodorant or antiperspirant compositions according to the invention are characterized in that the at least one water-soluble polyhydric C₂-C₉ alkanol with 2-6 hydroxyl groups and/or at least one water-soluble polyethylene glycol with 3-20 ethylene oxide units is contained in total in quantities of 3-30 wt. %, preferably of 8-25 wt. %, particularly preferably of 10-18 wt. %, in each case relative to the total composition.

Preferred compositions according to the invention contain water. The proportion of water preferably amounts to 0.1-99 wt. %, particularly preferably to 3 to 80 wt. %, extremely preferably to 10 to 70 wt. %, furthermore preferably to 15-60 wt. %, 20-50 wt. %, 30-40 wt. %, in each case relative to the total composition.

Particularly preferred deodorant or antiperspirant compositions according to the invention, in particular those in stick form, are characterized in that at least one wax component with a melting point in the range from 25-<50° C., selected from coconut oil fatty acid glycerol mono-, di- and triesters, Butyrospermum parkii (shea butter) and esters of saturated, monovalent C₈-C₁₈ alcohols with saturated C₁₂-C₁₈ monocarboxylic acids and mixtures of these substances, is contained. These lower-melting wax components permit optimization of the consistency of products in stick or cream form and minimization of visible residues on the skin. Particularly preferred substances are commercial products with the INCI name cocoglycerides, in particular the commercial products Novata® (from Cognis), particularly preferably Novata® AB, a mixture of C₁₂-C₁₈ mono-, di- and triglycerides, which melts in the range from 30-32° C., and the products of the Softisan range (Sasol Germany GmbH) with the INCI name hydrogenated cocoglycerides, in particular Softisan 100, 133, 134, 138, 142. Further preferred esters of saturated, monovalent C₁₂-C₁₈ alcohols with saturated C₁₂-C₁₈ monocarboxylic acids are stearyl laurate, cetearyl stearate (for example Crodamol® CSS), cetyl palmitate (for example Cutina® CP) and myristyl myristate (for example Cetiol® MM).

Further particularly preferred deodorant or antiperspirant compositions according to the invention, in particular those in stick form, are characterized in that the at least one wax component with a melting point in the range from 25-<50° C. is contained in quantities of 0.01 to 20 wt. %, preferably of 3-20 wt. %, particularly preferably of 5-18 wt. % and extremely preferably of 6-15 wt. %, relative to the total composition.

Particularly preferred deodorant or antiperspirant compositions according to the invention are characterized in that, in order to improve stick consistency and organoleptic properties, they furthermore contain at least one solid, water-insoluble particulate filler. In one extremely preferred embodiment, this filler is selected from optionally modified starches (for example from maize, rice, potatoes) and starch derivatives which are, if desired, pregelatinized, in particular starch derivatives such as aluminum starch octenylsuccinate (for example DRY FLO®), sodium starch octenylsuccinate, cellulose and cellulose derivatives, silicon dioxide, silicas, for example Aerosil® grades, spherical polyalkylsesquisiloxane particles (in particular Aerosil® R972 and Aerosil® 200V from Degussa), silica gels, talcum, kaolin, clays, for example bentonites, magnesium-aluminum silicates, boron nitride, lactoglobulin derivatives, for example sodium C₈₋₁₆-isoalkylsuccinyl lactoglobulin sulfonate, from Brooks Industries obtainable as the commercial product Biopol® OE, glass powders, polymer powders, in particular of polyolefins, polycarbonates, polyurethanes, polyamides, for example nylon, polyesters, polystyrenes, polyacrylates, (meth)acrylate or (meth)acrylate-vinylidene copolymers, which may be crosslinked, or silicones, and mixtures of these substances.

Polymer powders based on a polymethacrylate copolymer are for example obtainable as the commercial product Polytrap® 6603 (Dow Corning). Other polymer powders, for example based on polyamides, are obtainable under the name Orgasol® 1002 (polyamide 6) and Orgasol® 2002 (polyamide 12) from Elf Atochem. Further polymer powders which are suitable for the purpose according to the invention are, for example, polymethacrylates (Micropearl® M from SEPPIC or Plastic Powder A from NIKKOL), styrene-divinylbenzene copolymers (Plastic Powder FP from NIKKOL), polyethylene and polypropylene powder (ACCUREL® EP 400 from AKZO) or also silicone polymers (Silicone Powder X2-1605 from Dow Corning). Particularly preferred deodorant or antiperspirant compositions according to the invention are characterized in that they contain at least one solid, water-insoluble particulate filler in a total quantity of 0.01 to 30 wt. %, preferably of 5-20 wt. %, particularly preferably of 8-15 wt. %, in each case relative to the total composition.

Particularly preferred deodorant or antiperspirant compositions according to the invention are characterized in that at least one fragrance component is furthermore contained therein.

Perfumes, perfume oils or perfume oil constituents may be used as the fragrance component. According to the invention, perfume oils or fragrances may be individual odoriferous compounds, for example synthetic products of the ester, ether, aldehyde, ketone, alcohol and hydrocarbon type. Odoriferous substance compounds of the ester type are for example benzyl acetate, phenoxyethyl isobutyrate, p-tert.-butylcyclohexyl acetate, linalyl acetate, dimethylbenzylcarbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, Floramat, Melusat and Jasmecyclat. Ethers include, for example, benzyl ethyl ether and ambroxan, aldehydes include, for example, linear alkanals having 8-18 C atoms, citral, citronellal, citronellyloxyacetaldehyde, cyclamen aldehyde, lilial and bourgeonal, ketones include, for example, ionones, alpha-isomethylionone and methyl cedryl ketone, alcohols include anethole, citronellol, eugenol, geraniol, linalool, phenylethyl alcohol and terpineol, hydrocarbons mainly include terpenes such as limonene and pinene. Preferably, however, mixtures of various odoriferous substances which together produce an attractive fragrance note are used.

Such perfume oils may also contain natural odoriferous mixtures, as are obtainable from plant sources, for example pine, citrus, jasmine, patchouli, rose or ylang-ylang oil. Likewise suitable are muscatel sage oil, camomile oil, clove oil, melissa oil, mint oil, cinnamon leaf oil, lime blossom oil, juniper berry oil, vetiver oil, olibanum oil, galbanum oil and labdanum oil and orange-blossom oil, neroli oil, orange peel oil and sandalwood oil.

If it is to be perceptible, an odoriferous substance must be volatile, wherein, in addition to the nature of the functional groups and the structure of the chemical compound, an important role is also played by molar mass. Most odoriferous substances accordingly have molar masses of up to approximately 200 Dalton, while molar masses of 300 Dalton and above tend to be the exception. Due to the differing volatility of odoriferous substances, the odor of a perfume or fragrance composed of two or more odoriferous substances varies over the course of vaporization, it being possible to subdivide odor impressions into “head or top note,” “heart or middle note” and “end note or dry-out”. Since odor perception largely also depends on odor intensity, the head note of a perfume or fragrance does not solely consist of highly volatile compounds, while the end note largely consists of less volatile, i.e. tenacious odoriferous substances. When formulating perfumes, more highly volatile odoriferous substances may, for example, be bound to certain fixatives, so preventing them from vaporizing rapidly. Accordingly, in the following classification of odoriferous substances into “more highly volatile” or “tenacious” odoriferous substances, no statement is made about odor impression nor about whether the corresponding odoriferous substance is perceived as a top or heart note.

Tenacious odoriferous substances which may be used for the purposes of the present invention are, for example, essential oils such as angelica root oil, anise oil, arnica blossom oil, basil oil, bay oil, bergamot oil, champak flower oil, silver fir oil, silver fir cone oil, elemi oil, eucalyptus oil, fennel oil, pine-needle oil, galbanum oil, geranium oil, ginger grass oil, guaiacwood oil, gurjun balsam oil, helichrysum oil, ho oil, ginger oil, iris oil, cajeput oil, calmus oil, camomile oil, camphor oil, canaga oil, cardamom oil, cassia oil, pine needle oil, copaiva balsam oil, coriander oil, spearmint oil, caraway oil, cumin oil, lavender oil, lemongrass oil, lime oil, mandarin oil, melissa oil, ambrette oil, myrrh oil, clove oil, neroli oil, niaouli oil, olibanum oil, orange oil, origanum oil, palmarosa oil, patchouli oil, Peru balsam oil, petitgrain oil, pepper oil, peppermint oil, pimento oil, pine oil, rose oil, rosemary oil, sandalwood oil, celery oil, spike oil, star anise oil, terpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronellol, lemon oil and cypress oil. Higher-boiling or solid odoriferous substances of natural or synthetic origin may, however, also be used for the purposes of the present invention as tenacious odoriferous substances or odoriferous substance mixtures, i.e. fragrances. These compounds include the compounds stated below and mixtures thereof: ambrettolide, α-amylcinnamaldehyde, anethole, anisaldehyde, anisyl alcohol, anisole, anthranilic acid methyl ester, acetophenone, benzyl acetone, benzaldehyde, benzoic acid ethyl ester, benzophenone, benzyl alcohol, benzyl acetate, benzyl benzoate, benzyl formate, benzyl valerate, borneol, bornyl acetate, a-bromostyrene, n-decylaldehyde, n-dodecylaldehyde, eugenol, eugenol methyl ether, eucalyptol, farnesol, fenchone, fenchyl acetate, geranyl acetate, geranyl formate, least one N—C₂-C₂₄ acyl derivative and/or at least one C₂-C₂₄ ester and/or at least one physiologically acceptable salt of these protein active ingredients, and mixtures of these protein active ingredients, they contain at least one further skin-soothing active ingredient.

It has surprisingly been found that the skin-soothing action of known active ingredients could be increased and improved to an unexpected extent by yoghurt protein(s)/protein hydrolysate(s) and the derivatives thereof.

Skin-soothing active ingredients which are preferred according to the invention are selected from allantoin, α-bisabolol, α-lipoic acid, extracts from Centella asiatica, for example obtainable under the name Madecassicoside from DSM, glycyrrhetinic acid, which is particularly preferably present in encapsulated form in liposomes, and is obtainable in this form for example from Soliance under the trade name Calmsphere, mixtures of cereal waxes, extracts from shea butter and Argania spinosa oil with the INCI name “spent grain wax and Butyrospermum parkii (shea butter) extract and Argania spinosa kernel oil”, as are for example obtainable from Pentapharm under the tradename Stimu-Tex AS, extracts from Vanilla tahitensis, as are for example obtainable from Solabia under the tradename Vanirea (INCI: Vanilla tahitensis fruit extract), algin hydrolysates, as are for example obtainable from Codif under the tradename Phycosaccharide, in particular Phycosaccharide Al, extracts from Bacopa monniera, as are for example obtainable from Sederma under the tradename Bacocalmine, extracts from the rooibos plant, as are for example obtainable from Cosmetochem under the trade name Rooibos Herbasec MPE, yeast extracts, particularly preferably the commercial product Drieline (INCI name “sorbitol, yeast extract”), obtainable from Lanatech, the physiologically acceptable salts of sterol sulfates, as are for example obtainable from Vincience under the trade name Phytocohesine (INCI: sodium beta-sitosterylsulfate), aminodicarboxylic acids with a C-chain length of 3-6 carbon atoms and the physiologically acceptable salts thereof, preferably selected from aminomalonic acid, aminosuccinic acid (=aspartic acid), aminoglutaric acid and aminoadipic acid and the physiologically acceptable salts thereof such as heliotropin, methyl heptine carbonate, heptaldehyde, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl N-amyl ketone, methylanthranilic acid methyl ester, p-methylacetophenone, methylchavicol, p-methylquinoline, methyl-naphthyl ketone, methyl n-nonylacetaldehyde, methyl n-nonyl ketone, muscone, β-naphthol ethyl ether, β-naphthol methyl ether, nerol, nitrobenzene, n-nonylaldehyde, nonyl alcohol, n-octylaldehyde, p-oxyacetophenone, pentadecanolide, β-phenylethyl alcohol, phenylacetaldehyde dimethyl acetal, phenylacetic acid, pulegone, safrole, isoamyl salicylate, methyl salicylate, hexyl salicylate, cyclohexyl salicylate, santalol, skatole, terpineol, thymene, thymol, γ-undecalactone, vanillin, veratrumaldehyde, cinnamaldehyde, cinnamyl alcohol, cinnamic acid, ethyl cinnamate, benzyl cinnamate.

More highly volatile odoriferous substances include in particular lower-boiling odoriferous substances of natural or synthetic origin, which may be used alone or in mixtures. Examples of more highly volatile odoriferous substances are alkyl isothiocyanates (alkyl mustard oils), butanedione, limonene, linalool, linalyl acetate and propionate, menthol, menthone, methyl n-heptenone, phellandrene, phenylacetaldehyde, terpinyl acetate, citral, citronellal.

Particularly preferred deodorant or antiperspirant compositions according to the invention are characterized in that at least one fragrance component is contained in a total quantity of 0.00001 to 4 wt. %, preferably of 0.5-2 wt. %, in each case relative to the total composition.

Further cosmetic or dermatological compositions which are preferred according to the invention are characterized in that, in addition to the binary combination of a) at least one deodorant or antiperspirant active ingredient and b) at least one active ingredient selected from at least one yoghurt protein and/or at least one hydrolysate of a yoghurt protein and/or at potassium aspartate and magnesium aspartate, extracts from the seeds of Cucurbita pepo (zucchini), extracts from Mentha piperita, in particular from the leaves of Mentha piperita, N-acylated alkanolamines, in particular N-palmitoylethanolamine, hydrolysates and/or extracts from the alga Enteromorpha compressa (flat green nori, a green alga), in particular from the plant body (thallus) of the alga, commercially obtainable for example under the trade name Enteline 2, furthermore selected from the dipeptide Tyr-Arg, the esters thereof and the N-acyl derivatives thereof, in particular N-acetyl-Tyr-Arg hexyldecyl ester (for example Calmosensine from Sederma), and any desired mixtures of these substances.

Extracts from the seeds of Cucurbita pepo (zucchini) which are particularly preferred according to the invention are for example contained in the commercial products Curbilene (from Indena SpA, INCI: Cucurbita pepo (pumpkin) seed extract), Ocaline (from Soliance, INCI: sea water (and) water (and) Cucurbita pepo (pumpkin) seed extract), ARP 100 (from Greentech, INCI: water, alcohol, Serenoa serrulata fruit extract, Epilobium angustifolium extract, Cucurbita pepo (pumpkin) seed extract), ARP 100 Huileux (from Greentech, INCI: caprylic/capric triglyceride, Serenoa serrulata fruit extract, Epilobium angustifolium flower/leaf/stem extract, Cucurbita pepo (pumpkin) seed extract), cucurbitine (from Parfums Christian Dior, INCI: water (and) Cucurbita pepo (pumpkin) seed extract), Herbasol Extract Pumpkin (from Cosmetochem, INCI: water (and) propylene glycol (and) Cucurbita pepo (pumpkin) seed extract) and Pumpkin Extract (from Draco Natural Products, INCI: water (and) Cucurbita pepo (pumpkin) seed extract).

Extracts from Mentha piperita, in particular from the leaves of Mentha piperita, which are particularly preferred according to the invention are for example contained in the commercial products Calmiskin OP (from Silab, INCI: water (and) Mentha piperita (peppermint) leaf extract) and Caomint (from Solabia, INCI: propylene glycol, water, Mentha piperita (peppermint) leaf extract, Theobroma cacao (cocoa) extract). Allantoin is extremely preferred according to the invention; compositions which are extremely preferred according to the invention contain at least one deodorant or antiperspirant active ingredient, at least one yoghurt protein and/or at least one hydrolysate of a yoghurt protein and/or at least one N—C₂-C₂₄ acyl derivative and/or at least one C₂-C₂₄ ester and/or at least one physiologically acceptable salt of these protein active ingredients, and allantoin.

The further skin-soothing active ingredient (in addition to the at least one active ingredient selected from at least one yoghurt protein and/or at least one hydrolysate of a yoghurt protein and/or at least one N—C₂-C₂₄ acyl derivative and/or at least one C₂-C₂₄ ester and/or at least one physiologically acceptable salt of these protein active ingredients, and mixtures of these protein active ingredients) is preferably contained in a total quantity of 0.001 to 5 wt. %, particularly preferably of 0.01 to 2 wt. % and extremely preferably of 0.05 to 1 wt. %, in each case relative to the total composition.

In a further preferred embodiment, the compositions according to the invention contain at least one substance which inhibits hair growth. Preferred substances which inhibit hair growth are in particular selected from eflornithine, active ingredient combinations of soy protein hydrolysate, urea, menthol, salicylic acid and extracts from Hypericum perforatum, Hamamelis virginiana, Arnica montana and the bark of Salix alba, as are contained for example and preferably in the raw material Pilinhib® Veg LS 9109 from Laboratoires Serobiologiques with the INCI declaration “propylene glycol, hydrolysed soy protein, Hypericum perforatum extract, Hamamelis virginiana extract, Arnica montana flower extract, urea, Salix alba bark extract, menthol, salicylic acid”, furthermore active ingredient combinations of extracts from Epilobium angustifolium, the seeds of Cucurbita pepo (pumpkin, zucchini) and the fruits of Serenoa serrulata, as are for example and preferably contained in the raw materials ARP 100 from Greentech S. A./Rahn with the INCI declaration “water, alcohol, Serenoa serrulata fruit extract, Epilobium angustifolium extract, Cucurbita pepo (pumpkin) seed extract” and ARP 100 Huileux (from Greentech, INCI: caprylic/capric triglyceride, Serenoa serrulata fruit extract, Epilobium angustifolium flower/leaf/stem extract, Cucurbita pepo (pumpkin) seed extract), furthermore active ingredient combinations of xylitol and extracts of Citrus medica limonum (lemon) fruit, Carica papaya (papaya) and olive leaves, as are for example and preferably contained in the raw material Xyleine from Impag/Seporga with the INCI declaration “xylitol and Citrus medica limonum (lemon) fruit extract and Carica papaya (papaya) fruit extract and Olea europaea (olive) leaf extract”, furthermore active ingredient combinations of Humulus lupulus, Viscum album, Salvia officinalis, Carica papaya and Thuya occidentalis, as are for example and preferably contained in the raw material Plantafluid Komplex AH from Plantapharm with the INCI declaration “aqua, propylene glycol, Humulus lupulus, Viscum album, Salvia officinalis, Carica papaya, Thuya occidentalis”, and extracts from Larrea divaricata, as are for example and preferably contained in the raw material Capislow from Sederma which contains lecithin vesicles with a hydroglycolic extract of Larrea divaricata.

Further preferred hair growth-inhibiting active ingredients are selected from substances which inhibit protein tyrosine kinase, in particular from lavendustin A, erbstatin, tyrphostin, piceatannol, 4-hydroxybenzylidene-malononitrile, 3,5-di-tert.-butyl-4-hydroxybenzylidenemalononitrile, α-cyano-(3,4-dihydroxy)cinnamonitrile, α-cyano-(3,4,5-trihydroxy)cinnamonitrile, α-cyano-(3,4-dihydroxy)cinnamide, α-cyano-(3,4-dihydroxy)thiocinnamide, 2-amino-4-(4′-hydroxyphenyl)-1,1,3-tricyanobuta-1,3-diene, 2-amino-4-(3,4,5′-trihydroxyphenyl)-1,1,3-tricyanobuta-1,3-diene, 2-amino-4-(1H-alpha-indol-5-yl)-1,1,3-tricyanobuta-1,3-diene, 4-hydroxy-3-methoxy-5-(benzothiazolyl-thiomethyl)benzylidenecyanoacetamide, 4-amino-N-(2,5-dihydroxybenzyl)-methyl benzoate, α-cyano-(3,4-dihydroxy)-cinnamonitrile, 4-(3-chloroanilino)-6,7-dimethoxyquinazoline, α-cyano-(3,4-dihydroxy)-N-benzylcinnamide, (−)-R—N-(α-methylbenzyl)-3,4-dihydroxybenzylidenecyanoacetamide, α-cyano-(3,4-dihydroxy)-N-(3-phenylpropyl)cinnamide, α-cyano-(3,4-dihydroxy)-N-phenyl-cinnamide, α-cyano-(+)-(S)—N-(alpha-phenethyl)-(3,4-dihydroxy)cinnamide, α-cyano-(3,4-dihydroxy)-N-(phenylbutyl)cinnamide, herbimycin A, thiazolidinedione, phenazocine, 2,3-dihydro-2-thioxo-1H-indole-3-alkanoic acids, 2,2′-dithiobis-(1H-indole-3-alkanoic acids), sulfonylbenzoyl nitrostyrene, methyl caffeate, HNMPA(AM)₃(hydroxy-2-naphthalenylmethylphosphonic acid tris-acetoxymethyl ester) and N-acetyl-Asp-Tyr-(2-malonyl)-Val-Pro-Met-Leu-NH₂.

Further preferred hair growth-inhibiting active ingredients are selected from the substances which are disclosed in WO 2006/130330 A2, namely agonists of the farnesoid X receptor, preferably selected from bile acids, such as in particular lithocholic acid, cholic acid, deoxycholic acid, chenodeoxycholic acid, ursodeoxycholic acid and 6-alpha-ethylchenodeoxy-cholic acid, furthermore from farnesoids, in particular farnesol (3,7,11-trimethyl-2,6,10-dodecatrien-1-ol), farnesal, farnesyl acetate, 3,7,11-trimethyl-2,6,10-dodecatriene-1-carboxylic acid, methyl farnesyl ether, methyl farnesoate, ethyl farnesyl ether, ethyl farnesoate, furthermore from 7-methyl-9-(3,3-dimethyl-oxivanyl)-3-methyl-2,6-nonadienoic acid methyl ester juvenile hormone III), 7-methyl-9-(3,3-dimethyloxivanyl)-3-methyl-2,6-nonadienoic acid ethyl ester, 3-alpha,7-alpha-dihydroxy-6-alpha-ethyl-5p-cholan-24-oic acid, 7-alpha-dihydroxy-6-alpha-propyl-5p-cholan-24-oic acid, 7-alpha-dihydroxy-6-alpha-allyl-5p-cholan-24-oic acid, N-(2,2,2-trifluoroethyl)-N-[4-[2,2,2-trifluoro-1-hydroxy-1-(trifluoromethyl)-ethyl]phenyl]-benzene sulfone anilide, 3-[2-[2-chloro-4-[3-(2,6-dichlorophenyl)-5-(1-methylethyl)-4-isoxazolyl]methoxy]-phenylethenyl]-benzoic acid, [3,5-bis(1,1-dimethylethyl)-4-hydroxyphenyl]-ethenylidene]bisphosphonic acid tetraethyl ester, [2-[3,5-bis(1,1-dimethylethyl)-4-hydroxyphenyl]ethylidene]bisphosphonic acid tetrakis(1-methylethyl) ester, [2-[3,5-bis(1,1-di-methylethyl)-4-hydroxyphenyl]ethylidene]bisphosphonic acid tetraethyl ester and [3,5-bis(1,1-dimethylethyl-4-hydroxyphenyl]-ethenylidene]bisphosphonic acid tetrakis(1-methylethyl) ester.

Preferred compositions according to the invention contain at least one hair growth-inhibiting substance in a total quantity of 0.0001-5 wt. %, preferably of 0.001-2 wt. %, particularly preferably of 0.01-1 wt. %, and extremely preferably of 0.1-0.5 wt. %, in each case relative to the weight of active substance of the hair growth-inhibiting active ingredient(s) and the total weight of the composition according to the invention.

While particular embodiments of the present invention have been illustrated and described, it would be obvious to those skilled in the art that various other changes and modifications can be made without departing from the spirit and scope of the invention.

Other than where otherwise indicated, or where required to distinguish over the prior art, all numbers expressing quantities of ingredients herein are to be understood as modified in all instances by the term “about”. As used herein, the words “may” and “may be” are to be interpreted in an open-ended, non-restrictive manner. At minimum, “may” and “may be” are to be interpreted as definitively including, but not limited to, the composition, structure, or act recited.

As used herein, and in particular as used herein to define the elements of the claims that follow, the articles “a” and “an” are synonymous and used interchangeably with “at least one” or “one or more,” disclosing or encompassing both the singular and the plural, unless specifically defined herein otherwise. The conjunction “or” is used herein in both in the conjunctive and disjunctive sense, such that phrases or terms conjoined by “or” disclose or encompass each phrase or term alone as well as any combination so conjoined, unless specifically defined herein otherwise.

The description of a group or class of materials as suitable or preferred for a given purpose in connection with the invention implies that mixtures of any two or more of the members of the group or class are equally suitable or preferred. Description of constituents in chemical terms refers unless otherwise indicated, to the constituents at the time of addition to any combination specified in the description, and does not necessarily preclude chemical interactions among the constituents of a mixture once mixed. Steps in any method disclosed or claimed need not be performed in the order recited, except as otherwise specifically disclosed or claimed.

Changes in form and substitution of equivalents are contemplated as circumstances may suggest or render expedient. Although specific terms have been employed herein, such terms are intended in a descriptive sense and not for purposes of limitation.

The following Examples further illustrate the preferred embodiments within the scope of the present invention, but are not intended to be limiting thereof. It is understood that the examples and embodiments described herein are for illustrative purposes only and that various modifications or changes in light thereof will be suggested to one skilled in the art without departing from the scope of the present invention. The appended claims therefore are intended to cover all such changes and modifications that are within the scope of this invention.

### Exemplary Embodiments

The following Examples of formulations are intended to illustrate the subject matter of the invention without limiting it thereto.

Antiperspirant sticks according to the invention based on an oil-in-water emulsion (quantities stated in wt. %)

| | Example no. | | | | | | |
|---|---|---|---|---|---|---|---|
| Component | 1 | 2 | 3 | 4 | 5 | 6 | 7 |
| Cutina ®AGS | 2.5 | 2.5 | — | — | — | 2.5 | 2.5 |
| Cutina ® EGMS | — | — | 2.5 | 2 | — | — | — |
| Cutina ® PES | — | — | — | — | 2 | — | — |
| Cutina ® FS45 | 3.5 | 3.5 | 3.5 | 3.5 | 3.5 | 3.5 | 3.5 |
| Eumulgin ® B2 | 0.8 | 0.8 | 0.8 | 0.8 | 0.8 | 0.8 | 0.8 |
| Eumulgin ® B3 | 0.8 | 0.8 | 0.8 | 0.8 | 0.8 | 0.8 | 0.8 |
| Diisopropyl adipate | 6 | 6 | 6 | 6 | 6 | 6 | 6 |
| (Ceraphyl ® 230) | | | | | | | |
| Novata ® AB | 4 | 4 | 4 | 4 | 4 | 4 | 4 |
| Cutina ® CP | 5 | 5 | 5 | 5 | 5 | 5 | 5 |
| Cutina ® HR | 4 | 4 | 4 | 4 | 4 | 4 | 4 |
| Kester Wax K62 | 5 | 5 | 5 | 5 | 5 | 5 | 5 |
| Locron ® L (ACH | 40 | 40 | 40 | 40 | 40 | 40 | 40 |
| solution, 50%) | | | | | | | |
| Talcum Pharma G | 10 | 10 | 10 | 10 | 10 | 10 | 10 |
| Perfume | 1.2 | 1.2 | 1.2 | 1.2 | 1.2 | 1.2 | 1.2 |
| Sensiva SC 50 | — | — | 0.6 | 0.6 | 0.6 | 0.6 | 0.6 |
| Tinogard Q | 0.025 | 0.05 | 0.025 | — | 0.1 | 0.1 | — |
| Tinogard AS | — | — | 0.025 | — | — | — | — |
| 1,2-Propanediol | 10 | 10 | 10 | 10 | 10 | 10 | 10 |
| Yoghurt protein* | 0.05 | 0.08 | 0.1 | 0.02 | 0.01 | 0.1 | 0.05 |
| Lactose | — | — | — | 0.1 | 0.01 | 0.1 | 0.05 |
| Glucose | — | — | — | 0.1 | — | — | — |
| Water, deionized | 7.125 | 7.07 | 6.45 | 6.88 | 6.98 | 6.3 | 6.5 |
| Total | 100.0 | 100.0 | 100.0 | 100.0 | 100.0 | 100.0 | 100.0 |

*obtainable from spray-dried yoghurt

### Example 8

### Antiperspirant Emulsion (O/W) According to the Invention

| | wt. % |
|---|---|
| INGREDIENTS | proportion |
| ALUMINUM-ZIRCONIUM | 23.7 |
| TETRACHLOROHYDREX GLY | |
| STEARETH-2 | 2.4 |
| STEARETH-21 | 1.6 |
| PARFUM | 1.2 |
| PPG-15 STEARYL ETHER | 0.5 |
| ALUMINUM STARCH OCTENYLSUCCINATE | 0.1 |
| TOCOPHERYL ACETATE | 0.5 |
| Yoghurt Protein GBU | 0.1 |
| AQUA | ad 100.0 |

On the first day after production, the emulsion according to Example 8 exhibited a viscosity of 2200 mPa·s, measured with a Brookfield viscosimeter, spindle RV 4, 20 s⁻¹, without Helipath, at 20° C. ambient temperature and 20° C. sample temperature; it was packaged in a roll-on applicator.

### Example 9

### Antiperspirant Emulsion (O/W) According to the Invention

| | wt. % |
|---|---|
| INGREDIENTS | proportion |
| ALUMINUM CHLOROHYDRATE | 20.00 |
| STEARETH-2 | 2.30 |
| STEARETH-21 | 1.50 |
| PARFUM | 1.00 |
| PPG-15 STEARYL ETHER | 0.50 |
| ALUMINUM STARCH OCTENYLSUCCINATE | 0.10 |
| Yoghurt Protein GBU | 0.05 |
| Isopropyl myristate | 0.10 |
| AQUA | ad 100.00 |

On the first day after production, the emulsion according to Example 9 exhibited a viscosity of 1700 mPa·s, measured with a Brookfield viscosimeter, spindle RV 4, 20 s⁻¹, without Helipath, at 20° C. ambient temperature and 20° C. sample temperature; it was packaged in a roll-on applicator.

### Anhydrous Surfactant-containing Antiperspirant Sticks (Values in Parts by Weight)

| | 10.1 | 10.2 | 10.3 | 10.4 | 10.5 | 10.6 | 10.7 | 10.8 | 10.9 |
|---|---|---|---|---|---|---|---|---|---|
| Silicone oil DC ® | 28 | 28 | 23 | 23 | 23 | 38 | 42 | 47 | 31 |
| 245 | | | | | | | | | |
| Eutanol ®G 16 | 10 | — | — | 15 | 10 | — | 10 | — | 10 |
| Cetiol ® OE | — | 10 | 15 | — | — | — | — | — | — |
| Ucon Fluid ® AP | 5 | 5 | 5 | 5 | 5 | 5 | 5 | 5 | 5 |
| Cutina ® HR | 6 | 6 | 6 | 6 | 6 | 6 | 2 | 5 | 6 |
| Lorol ® C18 | 20 | 20 | 20 | — | 20 | 20 | — | — | 20 |
| Lanette O | — | — | — | 20 | — | — | 10 | 12 | — |
| Eumulgin ® B 3 | 3 | 3 | 3 | 3 | 3 | 3 | 3 | 3 | — |
| Cutina ® E 24 PF | — | — | — | — | 5 | — | — | — | — |
| Aluminum | 20 | 20 | 20 | 20 | 20 | 20 | 20 | — | — |
| chlorohydrate | | | | | | | | | |
| Talcum | 7.9 | 7 | 7.9 | 7.9 | 7.95 | 7.8 | 7.9 | 27 | 26.5 |
| Isopulegol | 0.1 | — | — | — | — | — | — | — | — |
| Eugenyl glucoside | — | 1.0 | — | — | — | — | — | — | — |
| Zinc gluconate | — | — | — | 0.1 | — | — | — | — | — |
| Zinc | — | — | — | — | 0.05 | — | — | — | — |
| pyrrolidonecarboxylic | | | | | | | | | |
| acid | | | | | | | | | |
| Zinc cocoyl ether | — | — | — | — | — | 0.2 | — | — | — |
| sulfate | | | | | | | | | |
| Zinc glycinate | — | — | — | — | — | — | 0.1 | — | — |
| monohydrate | | | | | | | | | |
| Ascorbyl | — | — | — | — | — | — | — | 1.0 | — |
| glucoside | | | | | | | | | |
| 2- | — | — | — | — | — | — | — | — | 1.5 |
| Ethylhexylglycerol | | | | | | | | | |
| ether | | | | | | | | | |
| Yoghurt Protein | 0.05 | 0.1 | — | — | — | — | 0.06 | 0.07 | 0.09 |
| GBU | | | | | | | | | |
| Yoghurt protein* | — | — | 0.01 | 0.02 | 0.03 | 0.04 | — | — | — |
| Lactose | — | — | 0.05 | 0.05 | 0.03 | 0.04 | — | — | — |
| Glucose | — | — | 0.01 | — | — | — | — | — | — |
| Galactose | — | — | — | 0.02 | 0.01 | — | — | — | — |

*obtainable from spray-dried yoghurt

### Anhydrous Surfactant-containing Antiperspirant Sticks (Values in Weight-%)

| | 10.10 | 10.11 | 10.12 | 10.13 | 10.14 |
|---|---|---|---|---|---|
| PPG-14 butyl ether | 15.00 | 18.00 | 12.00 | 5.00 | 19.00 |
| Hydrogenated castor oil | 1.00 | 1.50 | 2.00 | 1.50 | 1.50 |
| Stearyl alcohol | 20.00 | 18.00 | 15.00 | 18.00 | 18.00 |
| Ceteareth-30 | 3.00 | 2.00 | 4.00 | 3.00 | — |
| Isoceteth-20 | — | — | — | — | 2.50 |
| Parfum | 1.00 | 1.20 | 0.80 | 1.10 | 1.50 |
| Aluminum chlorohydrate | 20.00 | 22.00 | 18.00 | 20.00 | — |
| Aluminum-zirconium | — | — | — | — | 22.00 |
| tetrachlorohydrate | | | | | |
| glycine | | | | | |
| Allantoin | 0.10 | — | — | — | 0.10 |
| Novata AB | 4.00 | 6.00 | 3.00 | 4.00 | 5.00 |
| Talcum | 3.00 | 2.00 | 5.00 | 3.00 | 3.00 |
| Tocopheryl acetate | 0.20 | | 0.50 | | 0.10 |
| Yoghurt Protein GBU | 0.05 | 0.1 | — | 0.05 | — |
| Yoghurt protein* | — | — | 0.01 | — | 0.02 |
| Lactose | — | — | 0.05 | — | 0.05 |
| Glucose | — | — | 0.01 | — | — |
| Galactose | — | — | — | — | 0.02 |
| Cyclopentasiloxane | ad 100 | ad 100 | ad 100 | ad 100 | ad 100 |

### Anhydrous Surfactant-containing Antiperspirant Sticks (Values in Weight-%)

| | 10.14 | 10.15 | 10.16 | 10.17 |
|---|---|---|---|---|
| Hexyldecanol | 10.00 | 12.00 | 10.00 | 8.00 |
| PPG-14 butyl ether | 6.00 | 5.00 | 6.00 | 8.00 |
| Hydrogenated castor oil | 4.00 | 5.00 | 6.00 | 5.00 |
| Stearyl alcohol | 12.00 | 14.00 | 11.00 | 16.00 |
| Cetyl alcohol | 6.00 | 5.00 | 6.00 | 3.00 |
| PEG-20 glyceryl stearate | 5.00 | 4.00 | 6.00 | 4.00 |
| Ceteareth-30 | 3.00 | 1.00 | 3.00 | |
| Parfum | 1.00 | 1.20 | 0.80 | 1.00 |
| Aluminum chlorohydrate | 20.00 | 20.00 | 18.00 | — |
| Aluminum-zirconium | — | — | — | 23.00 |
| tetrachlorohydrate glyc | | | | |
| Talcum | 8.00 | 5.00 | 8.00 | 7.00 |
| Tocopheryl acetate | 0.25 | — | 0.50 | — |
| Yoghurt Protein GBU | — | — | — | 0.06 |
| Yoghurt protein* | 0.02 | 0.03 | 0.04 | — |
| Lactose | 0.05 | 0.03 | 0.04 | — |
| Glucose | — | — | — | — |
| Galactose | 0.02 | 0.01 | — | — |
| Cyclopentasiloxane | ad 100 | ad 100 | ad 100 | ad 100 |

Anhydrous Antiperspirant Wax Sticks without Cyclomethicone (Values in wt. %)

| | 10.18 | 10.19 | 10.20 | 10.21 | 10.22 | 10.23 |
|---|---|---|---|---|---|---|
| Stearyl alcohol | 16 | 18 | 18 | 18 | 18 | 18 |
| Behenyl alcohol | — | — | 0.2 | — | — | — |
| Ceteareth-30 | 3 | 3 | 3 | 3 | 3 | 3 |
| PPG-14 butyl ether | 15 | 5 | 10 | 15 | 10 | 7.5 |
| Hydrogenated | — | — | — | — | 15 | 15 |
| polydecene | | | | | | |
| Coco glycerides | 5 | 4 | 4 | 4 | 4 | 4 |
| Hydrogenated | 1.5 | 1.5 | 2 | 1.5 | 1.5 | 1.5 |
| castor oil | | | | | | |
| Aluminum | 20 | 20 | 20 | 20 | 20 | 20 |
| chlorohydrate | | | | | | |
| Talcum | 5 | 3 | 5 | 3 | 3 | 5 |
| Yoghurt Protein | 0.1 | 0.08 | 0.05 | 0.1 | 0.06 | 0.1 |
| GBU | | | | | | |
| Parfum | 1 | 1 | 1 | 1 | 1 | 1 |
| Dimethicone (2 cSt) | — | — | 18.35 | 23 | ad 100 | ad 100 |
| Dimethicone (5 cSt) | ad 100 | ad 100 | 18.4 | 11.4 | — | — |

### Sprayable, Translucent Antiperspirant Microemulsion (Values in wt. %)

| | 11.1 | 11.2 | 11.3 | 11.4 | 11.5 | 11.6 | 11.7 | 11.8 | 11.9 |
|---|---|---|---|---|---|---|---|---|---|
| Plantaren ® 1200 | 1.71 | 1.71 | — | 1.71 | 1.71 | — | 1.71 | 1.71 | 1.71 |
| Plantaren ® 2000 | 1.14 | 1.39 | 2.40 | 1.14 | 1.39 | 2.40 | 1.14 | 1.39 | 1.39 |
| Glycerol | 0.71 | 0.71 | — | 0.71 | 0.71 | — | 0.71 | 0.71 | 0.71 |
| monooleate | | | | | | | | | |
| Dioctyl ether | 4.00 | 4.00 | 0.09 | 4.00 | 4.00 | 0.09 | 4.00 | 4.00 | 4.00 |
| Octyldodecanol | 1.00 | 1.00 | 0.02 | 1.00 | 1.00 | 0.02 | 1.00 | 1.00 | 1.00 |
| Perfume oil | 1.00 | 1.00 | 1.00 | 1.00 | 1.00 | 1.00 | 1.00 | 1.00 | 1.00 |
| Aluminum | 8.00 | 5.00 | 5.00 | — | — | — | 8.00 | 5.00 | 5.00 |
| chlorohydrate | | | | | | | | | |
| 1,2-Propylene | 5.00 | 5.00 | — | 5.00 | 5.00 | — | 5.00 | 5.00 | 5.00 |
| glycol | | | | | | | | | |
| Glycerol | — | — | 5.00 | — | — | 5.00 | — | — | — |
| Phenoxyethanol | 1.0 | — | — | — | 1.0 | 1.0 | — | — | — |
| Zinc lactate | — | 0.2 | — | — | — | — | — | — | — |
| Hinokitiol | — | — | 0.01 | — | — | — | — | — | — |
| Copper gluconate | — | — | — | 0.1 | — | — | — | — | — |
| Glycine | — | — | — | — | — | — | 0.2 | — | — |
| Phytic acid | — | — | — | — | — | — | — | 1.0 | — |
| Tripotassium | — | — | — | — | — | — | — | — | 1.0 |
| citrate | | | | | | | | | |
| Yoghurt Protein | 0.05 | 0.1 | — | — | — | — | 0.06 | 0.07 | 0.09 |
| GBU | | | | | | | | | |
| Yoghurt protein* | — | — | 0.01 | 0.02 | 0.03 | 0.04 | — | — | — |
| Lactose | — | — | 0.05 | 0.05 | 0.03 | 0.04 | — | — | — |
| Glucose | — | — | 0.01 | — | — | — | — | — | — |
| Galactose | — | — | — | 0.02 | 0.01 | — | — | — | — |
| Water | ad | ad | ad | ad | ad | Ad | ad | ad | ad |
| | 100 | 100 | 100 | 100 | 100 | 100 | 100 | 100 | 100 |

*obtainable from spray-dried yoghurt

### Sprayable Antiperspirant Microemulsion (Values in wt. %)

| | 11.10 | 11.11 | 11.12 | 11.13 |
|---|---|---|---|---|
| Aluminum chlorohydrate 50% | 30.00 | 40.00 | 35.00 | 40.00 |
| solution | | | | |
| Dicaprylyl ether | 10.00 | 10.00 | 8.00 | 9.00 |
| Glycerol 86% | 5.00 | 3.00 | 5.00 | 3.00 |
| Beheneth-10 | 3.30 | 4.00 | 3.50 | 4.00 |
| Cetearyl isononanoate | — | — | 4.00 | 5.00 |
| Hexyldecanol/hexyldecyl | 3.00 | 5.00 | — | — |
| laurate | | | | |
| Parfum | 1.00 | 0.80 | 1.20 | 1.00 |
| Chamomile extract | 0.20 | — | 0.50 | — |
| Polysorbate 20/linoleic acid | 0.20 | 0.20 | 0.50 | — |
| ALLANTOIN | 0.10 | — | — | 0.20 |
| Yoghurt Protein GBU | 0.1 | 0.1 | 0.05 | 0.02 |
| Aqua | ad 100 | ad 100 | ad 100 | ad 100 |

The above-listed sprayable antiperspirant microemulsions may be dispensed both with a propellant-free pump sprayer and with a propellant or applied onto a substrate, for example a nonwoven or a cellulose wipe and thus be used as an antiperspirant wipe.

### Soap-containing Sticks (Values in wt. %)

| | 12.1 | 12.2 | 12.3 | 12.4 | 12.5 | 12.6 | 12.7 |
|---|---|---|---|---|---|---|---|
| Ethanol | 22.5 | 22.5 | 22.5 | 22.5 | 22.5 | 22.5 | 22.5 |
| Cutina ® FS 45 | 4.4 | 4.4 | 4.4 | 4.4 | 4.4 | 4.4 | 4.4 |
| 1,3-Butanediol | 31.7 | 31.7 | 31.7 | 31.7 | 31.7 | 31.7 | 31.7 |
| 1,2-Propylene | 21.0 | 21.0 | 21.0 | 21.0 | 21.0 | 21.0 | 21.0 |
| glycol | | | | | | | |
| Eutanol ® G | 2.0 | 2.0 | 2.0 | 2.0 | 2.0 | 2.0 | 2.0 |
| Aethoxal ® B | 3.0 | 3.0 | 3.0 | 3.0 | 3.0 | 3.0 | 3.0 |
| Cremophor ® RH | 0.05 | 0.05 | 0.05 | 0.05 | 0.05 | 0.05 | 0.05 |
| 455 | | | | | | | |
| NaOH, 45% | 1.44 | 1.44 | 1.44 | 1.44 | 1.44 | 1.44 | 1.44 |
| Phenoxyethanol | 1.0 | 1.0 | 1.0 | 1.0 | 1.0 | 1.0 | 1.0 |
| Sensiva ® SC 50 | 0.3 | 0.3 | 0.3 | 0.3 | 0.3 | 0.3 | 0.3 |
| Perfume oil | 2.0 | 2.0 | 2.0 | 2.0 | 2.0 | 2.0 | 2.0 |
| Eugenyl glucoside | 1.0 | — | — | — | — | — | — |
| Dequest ® 2066 | — | — | — | 0.2 | — | — | — |
| Green tea extract | — | 0.015 | — | — | — | — | — |
| Yoghurt Protein | 0.1 | 0.1 | — | — | — | 0.04 | 0.1 |
| GBU | | | | | | | |
| Yoghurt protein* | — | — | 0.01 | 0.05 | 0.1 | — | — |
| Lactose | — | — | — | 0.05 | 0.1 | — | — |
| Glucose | | | | 0.1 | | 0.02 | |
| Galactose | — | — | 0.01 | — | 0.03 | — | 0.2 |
| Phenylethyl alcohol | — | — | — | — | — | — | 1.0 |
| Water, dist. | ad 100 | ad 100 | ad 100 | ad 100 | ad 100 | ad 100 | ad 100 |

*obtainable from spray-dried yoghurt

### Soap-containing Deodorant Sticks (Values in wt. %)

| | 12.8 | 12.9 | 12.10 | 12.11 |
|---|---|---|---|---|
| Alcohol denat. | 40.00 | 40.00 | 35.00 | 30.00 |
| 1,2-Propylene glycol | 30.32 | 32.32 | 32.32 | 38.00 |
| 1,3-Butylene glycol | 12.00 | 10.00 | 15.00 | 12.00 |
| Sodium palmitate | 3.10 | 3.50 | 2.80 | 3.10 |
| Sodium stearate | 3.10 | 3.50 | 2.80 | 3.10 |
| Glycerol 86% | 2.00 | 1.00 | 1.70 | — |
| PPG-5 Laureth-5 | 0.50 | 1.00 | 1.00 | 0.50 |
| (50 wt. % sodium hydroxide | (2) | (2) | (2) | (2) |
| solution, as required) | | | | |
| Parfum | 1.00 | 0.60 | 1.30 | 1.00 |
| Octyldodecanol | 1.00 | 0.50 | 1.00 | 0.70 |
| Phenoxyethanol | 1.00 | 0.50 | 1.00 | 0.50 |
| Ethylhexylglycerol | 0.50 | — | 0.30 | — |
| Tocopheryl acetate | 0.05 | — | 0.10 | 0.25 |
| PEG-40 hydrogenated | 0.02 | — | — | 0.10 |
| castor oil | | | | |
| Yoghurt Protein GBU | 0.1 | 0.1 | — | 0.05 |
| Yoghurt protein* | — | — | 0.01 | — |
| Lactose | — | — | — | — |
| Glucose | — | — | — | — |
| Galactose | — | — | 0.01 | — |
| Aqua | ad 100 | ad 100 | ad 100 | Ad 100 |

### Ethanol-free Soap Based Deodorant Sticks

| | 12.12 | 12.13 | 12.14 | 12.15 |
|---|---|---|---|---|
| PEG-8 | 40.00 | 45.00 | 50.00 | 46.00 |
| Sodium palmitate | 2.50 | 2.50 | 2.50 | 2.50 |
| Sodium stearate | 2.50 | 2.50 | 2.50 | 2.50 |
| 1,3-Butylene glycol | 5.00 | 2.00 | 3.00 | 4.00 |
| PEG-14 dimethicone | 1.00 | 2.00 | 1.50 | 1.50 |
| (50 wt. % sodium | (1.5) | (1.2) | (1.4) | (1.3) |
| hydroxide solution, as | | | | |
| required) | | | | |
| Phenoxyethanol | 1.00 | 2.00 | 0.50 | 1.00 |
| Parfum | 1.00 | 1.20 | 0.80 | 1.00 |
| Ethylhexylglycerol | 0.30 | | 0.30 | |
| Steareth-10 | 0.20 | | 0.20 | 0.20 |
| Glycyrrhiza glabra | 0.20 | 0.50 | | 0.30 |
| extract | | | | |
| Yoghurt Protein GBU | 0.10 | 0.1 | — | 0.05 |
| Aqua | ad 100 | ad 100 | ad 100 | ad 100 |

### Deodorant in Pump Atomizer (Values in wt. %)

| | 13.1 | 13.2 | 13.3 | 13.4 | 13.5 | 13.6 | 13.7 | 13.8 |
|---|---|---|---|---|---|---|---|---|
| Ethanol 96% (DEP | 55.0 | 55.0 | 55.0 | 55.0 | 55.0 | 55.0 | 55.0 | 55.0 |
| denatured) | | | | | | | | |
| Triethyl citrate | 3.0 | 3.0 | 3.0 | 3.0 | 3.0 | 3.0 | 3.0 | 3.0 |
| Cremophor ® RH | 0.5 | 0.5 | 0.5 | 0.5 | 0.5 | 0.5 | 0.5 | 0.5 |
| 455 | | | | | | | | |
| Eucarol ® AGE-EC | 1.0 | — | — | — | — | — | — | — |
| Eucarol ® AGE-ET | — | 0.2 | — | — | — | — | — | — |
| Eucarol ® AGE-SS | — | — | 1.0 | — | — | — | — | — |
| Dequest ® 2016D | — | — | — | — | 0.1 | — | — | — |
| Yoghurt Protein | 0.05 | 0.1 | — | — | — | — | 0.06 | 0.07 |
| GBU | | | | | | | | |
| Yoghurt protein* | — | — | 0.01 | 0.02 | 0.03 | 0.04 | — | — |
| Lactose | — | — | 0.05 | 0.05 | 0.03 | 0.04 | — | — |
| Glucose | — | — | 0.01 | — | — | — | — | — |
| Galactose | — | — | — | 0.02 | 0.01 | — | — | — |
| Perfume oil | 1.0 | 1.0 | 1.0 | 1.0 | 1.0 | 1.0 | 1.0 | 1.0 |
| Water | ad | ad | ad | ad | ad | ad | Ad | ad |
| | 100 | 100 | 100 | 100 | 100 | 100 | 100 | 100 |

*obtainable from spray-dried yoghurt

### Anhydrous Deodorant Spray (Values in wt. %)

| | 14.1 | 14.2 |
|---|---|---|
| 2-Octyldodecanol | 0.5 | 0.5 |
| Ethanol 99% (DEP denatured) | 39 | 39.45 |
| Naringin | 0.05 | |
| Hinokitiol | — | 0.01 |
| Yoghurt Protein GBU | 0.1 | 0.08 |
| n-Butane | ad 100 | ad 100 |

### Antiperspirant Roll-on (Values in wt. %)

| | 15.1 | 15.2 | 15.3 |
|---|---|---|---|
| Ethanol, 96% (DEP denatured) | 30.0 | 30.0 | 30.0 |
| Mergital ® CS 11 | 2.0 | 2.0 | 2.0 |
| Eumulgin ® B 3 | 2.0 | 2.0 | 2.0 |
| Aluminum chlorohydrate | 20.0 | 20.0 | 20.0 |
| Hydroxyethylcellulose | 0.5 | 0.5 | 0.5 |
| Dermosoft ® HMA | 0.5 | — | — |
| Yoghurt Protein GBU | 0.02 | 0.1 | 0.05 |
| Perfume oil | 0.8 | 0.8 | 0.8 |
| Water | ad 100 | ad 100 | ad 100 |

### Antiperspirant Spray, Suspension Type (Values in wt. %)

| | 16.1 | 16.2 | 16.3 | 16.4 | 16.5 |
|---|---|---|---|---|---|
| DC-245 | 10.0 | 10.0 | 10.0 | 10.0 | 10.0 |
| Isopropyl myristate | 5.0 | 5.0 | 5.0 | 5.0 | 5.0 |
| Aluminum chlorohydrate | 5.0 | 5.0 | 5.0 | 5.0 | 5.0 |
| powder | | | | | |
| Aerosil R 972 | 2.0 | 2.0 | 2.0 | 2.0 | 2.0 |
| Geraniol-7 EO | 0.5 | — | 0.5 | — | 0.5 |
| Alutrat ® | — | 2.0 | — | 2.0 | — |
| Yoghurt Protein GBU | 0.1 | — | — | — | 0.04 |
| Yoghurt protein* | — | 0.01 | 0.05 | 0.1 | — |
| Lactose | — | — | 0.05 | 0.1 | — |
| Glucose | — | — | 0.1 | — | 0.02 |
| Galactose | — | 0.03 | 0.02 | — | — |
| n-Butane | ad 100 | ad 100 | ad 100 | ad 100 | ad 100 |

*obtainable from spray-dried yoghurt

### Antiperspirant Wipes (Examples no. 17.1.-17.4)

For the embodiment according to the invention as an antiperspirant wipe, in each case 75 g of Example emulsions 11.1 or 11.2 or 2.3 per square meter or in each case 75 g of Example solutions 13.1 or 13.2 were applied onto a single ply substrate of 100% viscose with a basis weight of 50 g/m², cut into wipes of a suitable size and packaged in sachets.

### Water-in-oil Emulsion Sprays

| | Example 18.1 | | Example 18.2 | |
|---|---|---|---|---|
| | [wt. %, relative to | | [wt. %, relative | |
| | total | Example 18.1 | to total | Example 18.2 |
| | preparation | [wt. %, relative | preparation | [wt. %, relative |
| | incl. propellant | to W/O | incl. propellant | to W/O |
| Constituent | gas] | emulsion] | gas] | emulsion] |
| Dow Corning | 2.0 | 12.5 | 1.0 | 4.9 |
| 345 fluid | | | | |
| 1,2-Propylene | 0.5 | 3.1 | 2.0 | 9.8 |
| glycol | | | | |
| Dow Corning | 3.0 | 16.9 (oil) | 2.5 | 11.0 (oil) |
| 5225 C | | 1.9 | | 1.2 |
| | | (emulsifier) | | (emulsifier) |
| 2-Ethylhexyl | 0.5 | 3.1 | 0.5 | 2.4 |
| palmitate | | | | |
| Phenoxyethanol | 0.08 | 0.5 | 0.1 | 0.5 |
| Yoghurt Protein | 0.016 | 0.1 | 0.01 | 0.05 |
| GBU | | | | |
| Water, | 4.904 | 30.6 | 7.14 | 34.85 |
| deionized | | | | |
| Microdry | 5.0 | 31.3 | 7.25 | 35.3 |
| n-Butane | 84.0 | — | 79.5 | — |

### W/O Emulsion Compact Spray

| | | | Example | |
|---|---|---|---|---|
| | | | 19.2 | |
| | Example 19.1 | | [wt. %, | |
| | [wt. %, relative | | relative to | |
| | to total | | total | |
| | preparation | Example 19.1 | preparation | Example 19.2 |
| | incl. | [wt. %, relative | incl. | [wt. %, relative |
| | propellant | to W/O | propellant | to W/O |
| Constituent | gas] | emulsion] | gas] | emulsion] |
| Dow Corning 345 | 0.48 | 1.6 | 2.173 | 4.1 |
| fluid | | | | |
| 1,2-Propylene | 1.5 | 5.0 | 4.823 | 9.1 |
| glycol | | | | |
| Dow Corning | 4.2 | 12.53 | 7.42 | 12.53 |
| 5225 C | | (cyclomethicone) | | (cyclomethicone) |
| | | 1.47 | | 1.47 |
| | | (emulsifier) | | (emulsifier) |
| Benzoic acid | 1.38 | 4.6 | — | — |
| C12-15 alkyl | | | | |
| ester | | | | |
| 2-Ethylhexyl | — | — | 1.192 | 2.25 |
| palmitate | | | | |
| Parfum | 0.75 | 2.5 | — | — |
| Phenoxyethanol | 0.15 | 0.5 | 0.265 | 0.5 |
| Yoghurt Protein | 0.03 | 0.1 | 0.027 | 0.05 |
| GBU | | | | |
| Water, deionized | 1.71 | 5.7 | 2.12 | 4.0 |
| Aluminum | 19.8 | 66.0 | 34.98 | 66.0 |
| chlorohydrate, | | | | |
| 50% aqueous | | | | |
| solution | | | | |
| n-Butane | 70.0 | — | 47.0 | — |

Example composition 19.1 contains an antiperspirant emulsion according to the invention with 9.9 wt. % aluminum chlorohydrate, relative to the weight of the propellant gas-free emulsion. At a spraying rate of 0.35 g/s, 0.035 g of the antiperspirant active ingredient aluminum chlorohydrate is sprayed in a second onto the surface of the skin.

Example composition 19.2 contains an antiperspirant emulsion according to the invention with 17.49 wt. % aluminum chlorohydrate, relative to the weight of the propellant gas-free emulsion. At a spraying rate of 0.2 g/s, 0.035 g of the antiperspirant active ingredient aluminum chlorohydrate is sprayed in a second onto the surface of the skin.

### Deodorant Pump Spray/impregnating Solution for Deodorant Wipes

### Aqueous/alcoholic Solution of Solubilized Deodorant Active Ingredients and Perfume

| | 20.1 | 20.2 | 20.3 | 20.4 |
|---|---|---|---|---|
| Alcohol denat. | 50.00 | 55.00 | 60.00 | 40.00 |
| Triethyl citrate | 2.50 | 3.50 | 4.00 | 3.00 |
| PEG-40 hydrogenated | 1.00 | 0.50 | 0.50 | 2.00 |
| castor oil | | | | |
| Ethylhexylglycerol | 0.10 | 0.30 | — | — |
| Tocopheryl acetate | 0.05 | 0.20 | 0.10 | — |
| Benzophenone-2 | 0.01 | 0.01 | 0.01 | 0.05 |
| Colors approved for | 0.0001 | 0.0005 | 0.0010 | |
| cosmetics | | | | |
| Parfum | 0.80 | 1.00 | 2.00 | 1.50 |
| Yoghurt Protein GBU | 0.1 | 0.05 | 0.02 | 0.2 |
| Aqua | ad 100 | ad 100 | ad 100 | ad 100 |

### Clear Antiperspirant Gels

| | 21.1 | 21.2 | 21.3 | 21.4 |
|---|---|---|---|---|
| 1,2-Propylene glycol | 18.00 | 23.00 | 18.00 | 20.00 |
| Aluminum | 40.00 | 40.00 | 40.00 | 40.00 |
| chlorohydrate, 50% | | | | |
| solution | | | | |
| Cyclopentasiloxane | 14.20 | 14.20 | 14.20 | 14.20 |
| Alcohol denat. | 5.00 | 10.00 | 8.00 | 10.00 |
| Abil EM 97 | 3.50 | 2.50 | 3.20 | 3.00 |
| Parfum | 0.60 | 0.60 | 1.00 | 1.30 |
| Panthenol | 0.50 | — | — | — |
| Allantoin | — | 0.10 | — | — |
| Yoghurt Protein GBU | 0.1 | 0.05 | 0.02 | 0.2 |
| Aqua | ad 100 | ad 100 | ad 100 | ad 100 |

If clear gels are to be obtained, the refractive index of the aqueous phase must be adapted to the refractive index of the oil phase. Water or propylene glycol serve as the variable.

### Clear Deodorant Gels

| | 22.1 | 22.2 | 22.3 | 22.4 |
|---|---|---|---|---|
| Alcohol denat. | 30.00 | 40.00 | 50.00 | 60.00 |
| Ceteareth-12 | 1.50 | | 2.00 | |
| Ceteareth-30 | 2.50 | — | 2.00 | — |
| PEG-40 hydrogenated | | 3.00 | | 2.00 |
| castor oil | | | | |
| Carbomer | 0.30 | 0.50 | 0.80 | 1.00 |
| Neutralization agent | q.s. | q.s. | q.s. | q.s. |
| Parfum | 0.60 | 0.60 | 1.00 | 1.30 |
| Pilinhib VEG POE LS 9751 | 0.50 | — | 0.20 | — |
| Yoghurt Protein GBU | 0.1 | 0.05 | 0.02 | 0.2 |
| Aqua | ad 100 | ad 100 | ad 100 | ad 100 |

The thickener (Carbomer) must be adjusted to the desired pH with a suitable neutralizing agent (TEA, AMP, NaOH, LiOH).

### Antiperspirant Cream, Based on an Oil-in-water Emulsion

| | 23.1 | 23.2 | 23.3 | 23.4 |
|---|---|---|---|---|
| Aluminum chlorohydrate, | 40.00 | 40.00 | 35.00 | 45.00 |
| 50% solution | | | | |
| Glyceryl stearate | 5.00 | 4.50 | 5.50 | 6.00 |
| Cetyl alcohol | 2.00 | — | 3.00 | 1.50 |
| Behenyl alcohol | 1.50 | 4.00 | 3.50 | 5.00 |
| Dimethicone | 2.00 | 1.50 | 2.50 | 3.00 |
| Ceteareth-12 | 1.50 | 2.00 | 2.50 | 1.30 |
| Ceteareth-20 | 1.50 | 2.00 | 2.50 | 1.30 |
| Hexyldecanol/hexyldecyl | 3.00 | 4.00 | 2.50 | 2.40 |
| laurate | | | | |
| Cyclopentasiloxane | 1.50 | 3.00 | 2.00 | 1.00 |
| Tocopheryl acetate | 0.05 | 0.25 | — | — |
| Parfum | 0.80 | 1.00 | 1.50 | 2.00 |
| Allantoin | 0.10 | 0.10 | — | — |
| Preservative system | 0.05 | 0.50 | 0.50 | 0.50 |
| Yoghurt Protein GBU | 0.1 | 0.05 | 0.02 | 0.2 |
| Aqua | ad 100 | ad 100 | ad 100 | ad 100 |

### Antiperspirant Cream, Anhydrous (Soft Solid)

| | 24.1 | 24.2 | 24.3 | 24.4 |
|---|---|---|---|---|
| Aluminum chlorohydrate | 20.00 | 22.00 | 20.00 | — |
| Aluminum-zirconium | | | | 24.00 |
| tetrachlorohydrate glycine | | | | |
| Hexyldecanol | 5.00 | 4.50 | 5.50 | 6.00 |
| Dicaprylyl ether | 3.00 | 4.00 | 3.50 | 5.00 |
| Cocoglycerides | 5.00 | 6.00 | 7.00 | 3.00 |
| C18-C36 triglycerides | 6.00 | 5.00 | 4.00 | 3.00 |
| Ceteareth-30 | 3.00 | 2.00 | 2.50 | 4.00 |
| PEG-20 glycerylstearate | 5.00 | 6.00 | 3.00 | 2.00 |
| Cellulose | 3.00 | 2.00 | 5.00 | 1.00 |
| Aluminum starch | 5.00 | 4.00 | 6.00 | 5.00 |
| octenylsuccinate | | | | |
| Silica | 1.00 | 2.00 | 0.50 | |
| Talcum | 10.00 | 5.00 | 7.00 | 12.00 |
| Allantoin | 0.10 | 0.10 | | |
| Parfum | 1.00 | 1.50 | 2.00 | 0.80 |
| Pilinhib VEG POE LS 9751 | 0.50 | — | 0.20 | — |
| Yoghurt Protein GBU | 0.1 | 0.05 | 0.02 | 0.2 |
| Cyclopentasiloxane | ad 100 | ad 100 | ad 100 | ad 100 |

### Powder (Deodorant/antiperspirant)

| | 25.1 | 25.2 | 25.3 | 25.4 |
|---|---|---|---|---|
| Aluminum chlorohydrate | 20.00 | — | — | — |
| Aluminum-zirconium | | | | 24.00 |
| tetrachlorohydrate glycine | | | | |
| Silica | 2.00 | 2.00 | 1.00 | 1.00 |
| Triclosan | — | 0.30 | 0.10 | — |
| Sensiva SC 50 | — | — | 1.00 | — |
| Parfum | 1.00 | 0.50 | 2.00 | 1.00 |
| Yoghurt Protein GBU | 0.1 | 0.05 | 0.1 | 0.2 |
| Talcum | ad 100 | ad 100 | Ad 100 | ad 100 |

### Deodorant Soaps

| | 26.1 | 26.2 | 26.3 | 26.4 |
|---|---|---|---|---|
| Sodium tallowate | 55.00 | — | 60.00 | — |
| Sodium palmitate | — | 55.00 | — | 60.00 |
| Sodium cocoate | 22.00 | — | 27.00 | — |
| Sodium palm oleate | — | 22.00 | — | 27.00 |
| Talcum | 10.00 | 10.00 | — | — |
| Lauryl glucoside | — | 2.00 | — | 2.00 |
| Parfum | 1.00 | 1.00 | 1.50 | 0.50 |
| Sodium chloride | 0.50 | 0.50 | 0.50 | 0.50 |
| Tetrasodium EDTA | 0.30 | 0.20 | 0.30 | 0.10 |
| Tocopherol | 0.10 | 0.30 | — | — |
| Chlorhexidine digluconate | 0.30 | | 0.50 | — |
| Colors approved for cosmetics | 0.01 | 0.05 | 0.03 | — |
| Yoghurt Protein GBU | 0.1 | 0.05 | 0.1 | 0.1 |
| Aqua | ad 100 | ad 100 | Ad 100 | ad 100 |

### Deodorant Syndets

| | 27.1 | 27.2 | 27.3 | 27.4 |
|---|---|---|---|---|
| Sodium laureth sulfate | 30.00 | 30.00 | 25.00 | 30.00 |
| Sodium cocoyl isethionate | 15.00 | 12.00 | 20.00 | 15.00 |
| Disodium laureth | 10.00 | 15.00 | 10.00 | 8.00 |
| sulfosuccinate | | | | |
| Cetylstearyl alcohol | 10.00 | 12.00 | 10.00 | 10.00 |
| Stearic acid | 10.00 | 10.00 | 12.00 | 8.00 |
| Starch | 10.00 | 2.00 | 10.00 | 5.00 |
| Talcum | 2.00 | 10.00 | — | 5.00 |
| Parfum | 0.50 | 1.00 | 0.30 | 0.80 |
| Tetrasodium EDTA | 0.30 | 0.20 | 0.30 | 0.10 |
| Tocopheryl acetate | 0.10 | 0.30 | — | — |
| Sensiva SC 50 | 0.30 | 0.50 | 0.50 | — |
| Colors approved for cosmetics | 0.01 | 0.05 | 0.03 | — |
| Yoghurt Protein GBU | 0.1 | 0.05 | 0.1 | 0.1 |
| Aqua | ad 100 | ad 100 | Ad 100 | ad 100 |

### Deodorant Wash Lotions

| | 28.1 | 28.2 | 28.3 | 28.4 |
|---|---|---|---|---|
| Sodium laureth sulfate | — | 5.00 | 4.00 | — |
| Disodium laureth sulfosuccinate | 6.00 | | 2.00 | 8.00 |
| Lauryl glucoside | 4.00 | 5.00 | 4.00 | 4.00 |
| Potassium cocoyl hydrolysed | 2.00 | 3.00 | 5.00 | 2.00 |
| collagen | | | | |
| PEG-7 glyceryl cocoate | 3.00 | 3.00 | 5.00 | 3.00 |
| PEG-120 methyl glucose | 1.00 | 2.00 | 3.00 | 1.00 |
| dioleate | | | | |
| Parfum | 0.30 | 0.50 | 0.80 | 0.50 |
| Tetrasodium EDTA | 0.30 | 0.20 | — | 0.10 |
| Tocopheryl acetate | 0.10 | 0.30 | — | — |
| Sensiva SC 50 | 0.30 | — | 0.50 | — |
| Citric acid | 0.20 | 0.30 | 0.20 | 0.30 |
| Colors approved for cosmetics | 0.01 | 0.05 | 0.03 | — |
| Yoghurt Protein GBU | 0.1 | 0.05 | 0.1 | 0.1 |
| Aqua | ad 100 | ad 100 | Ad 100 | ad 100 |

The Example compositions according to the invention are applied onto the skin, in particular the armpit skin.

The present application accordingly also provides a method for the non-therapeutic treatment of the skin that is characterized in that a composition according to any one of claims 1-10 is applied onto the skin, in particular the armpit skin. Application onto the skin particularly preferably takes place after shaving.

The compositions according to the invention are distinguished by elevated skin compatibility which is improved relative to the prior art.

### List of Raw Materials Used

| | | Supplier/ |
|---|---|---|
| Component | INCI | manufacturer |
| Abil EM 97 | Bis-PEG/PPG-14/14 dimethicone | Goldschmidt |
| Aethoxal ® B | PPG-5 Laureth-5 | Cognis |
| Alutrat ® | Aluminum citrate | Vevy |
| Cetiol ® OE | DICAPRYLYL ETHER | Cognis |
| Cremophor ® RH 455 | Hydrogenated castor oil with 40 | BASF |
| | EO, propylene glycol-containing | |
| Cutina ® AGS | Glycol distearate | Cognis |
| Cutina ® EGMS | Glycol stearate | Cognis |
| Cutina ® PES | Pentaerythritol distearate | Cognis |
| Cutina ® CP | Cetyl palmitate | Cognis |
| Cutina ® FS45 | Palmitic acid, stearic acid | Cognis |
| Cutina ® HR | Hydrogenated castor oil | Cognis |
| Cutina ® E 24 PF | PEG-20 GLYCERYL STEARATE | Cognis |
| DC ® 245 | Cyclopentasiloxane | Dow Corning |
| Dequest ® 2016D | 1-Hydroxyethane-1,1- | Solutia |
| | diphosphonic acid tetrasodium | (Monsanto) |
| | salt, active substance 84% | |
| Dequest ® 2066 | Diethylenetriaminepenta(methylenephosphonic | Solutia |
| | acid) sodium salt, | (Monsanto) |
| | active acid 45-49% | |
| Dermosoft HMA | p-Hydroxymandelic acid sodium | Dr. Straetmans |
| | salt, active substance 95-96% | |
| Dow Corning 200 fluid 5 cSt | Dimethicone | Dow Corning |
| Dow Corning 345 fluid | Cyclomethicone | Dow Corning |
| | (decamethylcyclopentasiloxane, | |
| | dodecamethylcyclohexasiloxane) | |
| Dow Corning 5225 C | Cyclomethicone, PEG/PPG-18/18 | Dow Corning |
| Formulation Aid | dimethicone in the weight ratio | |
| | 9:1 | |
| Eucarol ® AGE-EC-UP | Disodium cocopolyglucose | Cesalpinia |
| | citrate, 30% active substance in | Chemicals |
| | water | |
| Eucarol ® AGE-ET-UP | Disodium cocopolyglucose | Cesalpinia |
| | tartrate, 30% active substance in | Chemicals |
| | water | |
| Eucarol ® AGE-SS | Disodium cocopolyglucose | Cesalpinia |
| | sulfosuccinate, 45% active | Chemicals |
| | substance in water | |
| Eumulgin ® B2 | Ceteareth-20 | Cognis |
| Eumulgin ® B3 | Ceteareth-30 | Cognis |
| Eumulgin ® B 3 | CETEARETH-30 | Cognis |
| Eutanol ® G | Octyldodecanol | Cognis |
| Eutanol ® G16 | 2-Hexyldecanol | Cognis |
| Green tea extract | Pulverulent | Dragoco |
| Kester Wax K62 | Cetearyl behenate | Koster Keunen |
| Lanette ® O | Cetyl/stearyl alcohol in the ratio | Cognis |
| | 1:1 | |
| Locron L (ACH solution, | Aluminum chlorohydrate | Clariant |
| 50%) | | |
| Lorol ® C18 | Stearyl alcohol | Cognis |
| Mergital ® CS 11 | CETEARETH-11 | Cognis |
| Microdry ® | Aluminum chlorohydrate | Reheis |
| Novata ® AB | Cocoglycerides | Cognis |
| Pilinhib VEG POE LS | PROPYLENE GLYCOL, | Laboratoires |
| 9751 | HYDROLYSED SOY PROTEIN, | Sérobiologiques |
| | HYPERICUM PERFORATUM | |
| | EXTRACT, HAMAMELIS | |
| | VIRGINIANA (WITCH HAZEL) | |
| | LEAF EXTRACT, ARNICA | |
| | MONTANA FLOWER EXTRACT, | |
| | UREA, SALIX ALBA (WILLOW) | |
| | BARK EXTRACT, MENTHOL, | |
| | SALICYLIC ACID | |
| Plantaren ® 1200 | LAURYL GLUCOSIDE, approx. | Cognis |
| | 50% active substance | |
| Plantaren ® 2000 | DECYL GLUCOSIDE, approx. | Cognis |
| | 50% active substance | |
| Sensiva ® SC 50 | 2-Ethylhexylglycerol ether | Schülke & Mayr |
| Tinogard AS | Bumetrizole | Ciba |
| Tinogard Q | Tris(tetramethylhydroxypiperidinol) | Ciba |
| | citrate | |
| Trilon A | Nitrilotriacetic acid 3 Na | BASF |
| Trilon B | EDTA 4 Na | BASF |
| Trilon ® M | Methylglycinediacetic acid | BASF |
| | trisodium salt, active substance | |
| | 39-41% | |
| Ucon Fluid ® AP | PPG-14 BUTYL ETHER | Amerchol |

What is claimed:

1. A cosmetic or dermatological deodorant or antiperspirant composition, comprising:
a) one or more deodorant or antiperspirant active ingredients; and
b) one or more yoghurt proteins, yoghurt protein derivatives, physiologically acceptable salts thereof, or any mixture thereof,
in a cosmetically or dermatologically acceptable carrier.

2. The composition of claim 1, wherein the yoghurt protein derivatives comprise one or more yoghurt protein hydrolysates, N—C₂-C₂₄ acyl derivatives, C₂-C₂₄ esters, or physiologically acceptable salts thereof.

3. The composition of claim 1, wherein b) comprises yoghurt, spray-dried yoghurt, freeze-dried yoghurt, lyophilized yoghurt, or yoghurt dried in another manner.

4. The composition of claim 1, comprising 0.001% to 0.1% by weight of b).

5. The composition of claim 1, comprising 3% to 25% by weight of the one or more antiperspirant active ingredients.

6. The composition of claim 1, comprising 0.1% to 10% by weight of the one or more deodorant active ingredients.

7. The composition of claim 1, further comprising at least one monosaccharide with 5 or 6 carbon atoms or at least one disaccharide.

8. The composition of claim 7, wherein the monosaccharide comprises glucose, fructose, galactose, arabinose, ribose, xylose, lyxose, allose, altrose, mannose, gulose, idose, talose, fucose, or rhamnose.

9. The composition of claim 7, wherein the disaccharide comprises sucrose, lactose, or trehalose.

10. The composition of claim 7, comprising 0.001% to 2.0% by weight of the monosaccharide with 5 or 6 carbon atoms or the disaccharide.

11. The composition of claim 1, wherein the cosmetically or dermatologically acceptable carrier comprises a powder, a stick form, an aerosol spray, a pump spray, a liquid or gel roll-on product, a cream, a lotion, a solution, a gel, or a substrate.

12. A method of improving skin compatibility or protective action of a cosmetic or dermatological deodorant or an antiperspirant composition or of reducing skin stinging, tightness, itching, or visible irritation caused by skin contact with the composition, comprising providing, in a cosmetically or dermatologically acceptable carrier:
a) one or more deodorant or antiperspirant active ingredients; and
b) one or more yoghurt proteins, yoghurt protein derivatives, physiologically acceptable salts thereof, or any mixture thereof.

13. The method of claim 12, wherein the yoghurt protein derivatives comprise one or more yoghurt protein hydrolysates, N—C₂-C₂₄ acyl derivatives, C₂-C₂₄ esters, or physiologically acceptable salts thereof.

14. The method of claim 12, wherein b) comprises yoghurt, spray-dried yoghurt, freeze-dried yoghurt, lyophilized yoghurt or yoghurt, dried in another manner.

15. The method of claim 12, wherein the cosmetic or dermatological deodorant or antiperspirant composition comprises 0.001% to 0.1% by weight of b).

16. The method of claim 12, wherein the cosmetic or dermatological deodorant or antiperspirant composition comprises 3% to 25% by weight of the antiperspirant active ingredient.

17. The method of claim 12, wherein the cosmetic or dermatological deodorant or antiperspirant composition comprises 0.1% to 10% by weight of the deodorant active ingredient.

18. The method of claim 12, wherein the cosmetic or dermatological deodorant or antiperspirant composition comprises a monosaccharide with 5 or 6 carbon atoms or a disaccharide.

19. The method of claim 18, wherein the monosaccharide comprises glucose, fructose, galactose, arabinose, ribose, xylose, lyxose, allose, altrose, mannose, gulose, idose, talose, fucose, or rhamnose.

20. The method of claim 18, wherein the disaccharide comprises sucrose, lactose, or trehalose.

21. The method of claim 18, wherein the cosmetic or dermatological deodorant or antiperspirant composition comprises 0.001% to 2.0% by weight of the monosaccharide with 5 or 6 carbon atoms or the disaccharide.

22. The method of claim 12, wherein the cosmetically or dermatologically acceptable carrier comprises a powder, a stick form, an aerosol spray, a pump spray, a liquid, a gel roll-on product, a cream, a lotion, a solution, a gel, or a substrate.

23. A method of non-therapeutic treatment of the skin, comprising applying to skin the composition of claim 1.

24. The method of claim 23, wherein the composition is applied to the skin after shaving.
